SEQUENCE LISTING (47 sequences)

SEQ ID NO: 1 - DNA, 1968 bp, synthetic construct
  source 1..1968: mol_type = other DNA; note = FMR1 isoform 1 (full length)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtacccggaggagggggctccggatacccctatgacgtcccggattatgcttacccatacgatgtgccggactatgct

SEQ ID NO: 2 - DNA, 1833 bp, synthetic construct
  source 1..1833: mol_type = other DNA; note = FMR1 Isoform 7
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 3 - DNA, 1758 bp, synthetic construct
  source 1..1758: mol_type = other DNA; note = FMR1 isoform 9
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 4 - DNA, 1782 bp, synthetic construct
  source 1..1782: mol_type = other DNA; note = FMR1 Isoform 17
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagaatacctccagtgaaggtagtcggctgcgcacgggtaaagatcgtaaccagaagaaagagaagccagacagcgtggatggtcagcaaccactcgtgaatggagtaccc

SEQ ID NO: 5 - DNA, 2063 bp, synthetic construct
  source 1..2063: mol_type = other DNA; note = FMR1 Isoform 7 + intron 16 (230bp)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 6 - DNA, 2233 bp, synthetic construct
  source 1..2233: mol_type = other DNA; note = FMR1 isoform 7 + intron 16 (400bp)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaattaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 7 - DNA, 2583 bp, synthetic construct
  source 1..2583: mol_type = other DNA; note = FMR1 isoform 7 + intron 16 (750bp)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaatatgcagtctgagtatttgtgaagaccttgacagccacactatgattttattatttattatttatttttatttttttgagacgggagtttcgcttttgttgcccaggctggagtgcaatggcgcaatctcggctcagagcaacctctgcctcccgggttcaagtgattctcctgccactaattttattttaactctagtgttagaggcatgtatgtcttctggtaatcagtaatagtaattcattttacctgtttgtttctcactgttttattctacttagaagaagacatgataggattgtgagtttttctctaactttggatgcagtgagatgaccagtgtgttccagttaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 8 - DNA, 2833 bp, synthetic construct
  source 1..2833: mol_type = other DNA; note = FMR1 isoform 7 + intron 16 (1000bp)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaatatgcagtctgagtatttgtgaagaccttgacagccacactatgattttattatttattatttatttttatttttttgagacgggagtttcgcttttgttgcccaggctggagtgcaatggcgcaatctcggctcagagcaacctctgcctcccgggttcaagtgattctcctgcctcagcctcctgagtagctggaattacaggcatccactaccatgcccagctaatttttgtatttttagtagagacggggtttcaccatgttggtcaggctggtcttgaactcccaacctcaggtgagatcatacctgtgatagatataaagatgtaaattaaagtctagttagtgagttatttgagggtaaagccagaataggaaaacattttgtttctcttttcctgggcttcacaatctttggagaataactaattttattttaactctagtgttagaggcatgtatgtcttctggtaatcagtaatagtaattcattttacctgtttgtttctcactgttttattctacttagaagaagacatgataggattgtgagtttttctctaactttggatgcagtgagatgaccagtgtgttccagttaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 9 - DNA, 2463 bp, synthetic construct
  source 1..2463: mol_type = other DNA; note = FMR1 isoform 7 + intron 14 (230bp) + intron 16 (400bp)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggtacaaactaagcattttactcagtaactttatctgttcctagacttatagctgctaatctctaatattcattagaaccccattataacaatttgccgctacatggtttccaatttataaattgggaagagttttggattacccctgaaacgtctctggaagcttctgttaaccctcttttttttttttttaaagtcagacaatggtatataacttttaactctcgataggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaattaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 10 - DNA, 2633 bp, synthetic construct
  source 1..2633: mol_type = other DNA; note = FMR1 isoform 7 + intron 14 (400bp) + intron 16 (400bp)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggtacaaactaagcattttactcagtaactttatctgttcctagacttatagctgctaatctctaatattcattagaaccccattataacaatttgccgctacatggtttccaattcacagtgggttaaattgtgttacaggagacatagagtaaaaacctgatgataaacacgtagagttcacagggccggtgcaagttcttaacctcaaatattgcaaagcccttcattttgagtttaaaattttgttttatgaaatggaaagtgataatactctttgtgggcttataaattgggaagagttttggattacccctgaaacgtctctggaagcttctgttaaccctcttttttttttttttaaagtcagacaatggtatataacttttaactctcgataggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaattaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 11 - DNA, 2983 bp, synthetic construct
  source 1..2983: mol_type = other DNA; note = FMR1 isoform 7 + intron 14 (750bp) + intron 16 (400bp)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggtacaaactaagcattttactcagtaactttatctgttcctagacttatagctgctaatctctaatattcattagaaccccattataacaatttgccgctacatggtttccaattcacagtgggttaaattgtgttacaggagacatagagtaaaaacctgatgataaacacgtagagttcacagggccggtgcaagttctgttcatgctctgattgggggtaaatctgcaaagcccttgtgtgttgatgattttcttaaggcctctcctgattttataactgtatagtcaaaacttttttcatttgtgtactgtggctcatttttattttgtcaggtatgtgctggtcatacttgctggatggactgtgtaaataggttttgtttatcagggtatcacacttaactgtcgtggataatcttttcctccagagagtatagatgtttaaaggaacacagctatttaatgatatggcacatcaaggtttgaacttaggtggctaataacatacctttttaaaaatgagaatgaaacatgtttataaccaaaattaacctcaaatattgcaaagcccttcattttgagtttaaaattttgttttatgaaatggaaagtgataatactctttgtgggcttataaattgggaagagttttggattacccctgaaacgtctctggaagcttctgttaaccctcttttttttttttttaaagtcagacaatggtatataacttttaactctcgataggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaattaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 12 - DNA, 3233 bp, synthetic construct
  source 1..3233: mol_type = other DNA; note = FMR1 isoform 7 + intron 14 (1000bp) + intron 16 (400bp)
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggtacaaactaagcattttactcagtaactttatctgttcctagacttatagctgctaatctctaatattcattagaaccccattataacaatttgccgctacatggtttccaattcacagtgggttaaattgtgttacaggagacatagagtaaaaacctgatgataaacacgtagagttcacagggccggtgcaagttctgttcatgctctgattgggggtaaatctgcaaagcccttgtgtgttgatgattttcttaaggcctctcctgattttataactgtatagtcaaaacttttttcatttgtgtactgtggctcatttttattttgtcaggtatgtgctggtcatacttgctggatggactgtgtaaatcaggggtctgcctcttgtggctcataaggtgattttgagtgacatggccaaagtcagccctaggcacagtcaagaagtaggcgaagtttctctttgccttcccagcttttctgtccgtgcttcttcaagagacccttccctgaagcagttgccatggtggcctcctttgccaccctctcagaaatgaccaaaatatatacattcccctctgtcctgggccaaatcttgctaaaaggagctctctttttagaggttttgtttatcagggtatcacacttaactgtcgtggataatcttttcctccagagagtatagatgtttaaaggaacacagctatttaatgatatggcacatcaaggtttgaacttaggtggctaataacatacctttttaaaaatgagaatgaaacatgtttataaccaaaattaacctcaaatattgcaaagcccttcattttgagtttaaaattttgttttatgaaatggaaagtgataatactctttgtgggcttataaattgggaagagttttggattacccctgaaacgtctctggaagcttctgttaaccctcttttttttttttttaaagtcagacaatggtatataacttttaactctcgataggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcaggtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaattaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 13 - protein, 632 aa, synthetic construct [sequence omitted]

SEQ ID NO: 14 - protein, 611 aa, synthetic construct
  source 1..611: note = FMRP protein isoform 7
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEEIMPPNSLPSNNSRVGPNAPEEKKHLDIKENSTHFSQPNSTKVQRGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQIRVDCNNERSVHTKTLQNTSSEGSRLRTGKDRNQKKEKPDSVDGQQPLVNGVP

SEQ ID NO: 15 - protein, 599 aa, synthetic construct
  source 1..599: note = FMRP protein isoform 8
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEEIMPPNSLPSNNSRVGPNAPEEKKHLDIKENSTHFSQPNSTKVQRGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSESDHRDELSDWSLAPTEEERESFLRRGDGRRRGGGGRGQGGRGRGGGFKGNDDHSRTDNRPRNPREAKGRTTDGSLQIRVDCNNERSVHTKTLQNTSSEGSRLRTGKDRNQKKEKPDSVDGQQPLVNGVP

SEQ ID NO: 16 - protein, 586 aa, synthetic construct
  source 1..586: note = FMRP protein isoform 9
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEEIMPPNSLPSNNSRVGPNAPEEKKHLDIKENSTHFSQPNSTKVQRGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSAPTEEERESFLRRGDGRRRGGGGRGQGGRGRGGGFKGNDDHSRTDNRPRNPREAKGRTTDGSLQIRVDCNNERSVHTKTLQNTSSEGSRLRTGKDRNQKKEKPDSVDGQQPLVNGVP

SEQ ID NO: 17 - protein, 594 aa, synthetic construct
  source 1..594: note = FMRP protein isoform 17
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEEIMPPNSLPSNNSRVGPNAPEEKKHLDIKENSTHFSQPNSTKVQRGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQNTSSEGSRLRTGKDRNQKKEKPDSVDGQQPLVNGVP

SEQ ID NO: 18 - protein, 582 aa, synthetic construct
  source 1..582: note = FMRP protein isoform 18
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEEIMPPNSLPSNNSRVGPNAPEEKKHLDIKENSTHFSQPNSTKVQRGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSESDHRDELSDWSLAPTEEERESFLRRGDGRRRGGGGRGQGGRGRGGGFKGNDDHSRTDNRPRNPREAKGRTTDGSLQNTSSEGSRLRTGKDRNQKKEKPDSVDGQQPLVNGVP

SEQ ID NO: 19 - protein, 569 aa, synthetic construct
  source 1..569: note = FMRP protein isoform 19
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEEIMPPNSLPSNNSRVGPNAPEEKKHLDIKENSTHFSQPNSTKVQRGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSAPTEEERESFLRRGDGRRRGGGGRGQGGRGRGGGFKGNDDHSRTDNRPRNPREAKGRTTDGSLQNTSSEGSRLRTGKDRNQKKEKPDSVDGQQPLVNGVP

SEQ ID NO: 20 - DNA, 20 bp, synthetic construct
  source 1..20: mol_type = other DNA; note = forward primer exon 14 FMR1 to confirm splicing
ggtcaaggaatgggtcgagg

SEQ ID NO: 21 - DNA, 20 bp, synthetic construct
  source 1..20: mol_type = other DNA; note = reverse primer exon17/HA-tag, overlap to confirm splicing. Exogenous FMR1 only
ctcctccgggtactccattc

SEQ ID NO: 22 - DNA, 22 bp, synthetic construct
  source 1..22: mol_type = other DNA; note = forward primer qPCR specific to iso17
aggctaaaggaagaacaacaga

SEQ ID NO: 23 - DNA, 27 bp, synthetic construct
  source 1..27: mol_type = other DNA; note = qPCR probe specific to alternatively spliced exon 17 (iso17)
tggatcccttcagaatacctccagtga

SEQ ID NO: 24 - DNA, 20 bp, synthetic construct
  source 1..20: mol_type = other DNA; note = forward primer qPCR specific to iso17
ctcctccgggtactccattc

SEQ ID NO: 25 - DNA, 230 bp, synthetic construct
  source 1..230: mol_type = other DNA; note = artificial intron 14 - 230bp
gtacaaactaagcattttactcagtaactttatctgttcctagacttatagctgctaatctctaatattcattagaaccccattataacaatttgccgctacatggtttccaatttataaattgggaagagttttggattacccctgaaacgtctctggaagcttctgttaaccctcttttttttttttttaaagtcagacaatggtatataacttttaactctcgatag

SEQ ID NO: 26 - DNA, 400 bp, synthetic construct
  source 1..400: mol_type = other DNA; note = artificial intron 14 - 400bp
gtacaaactaagcattttactcagtaactttatctgttcctagacttatagctgctaatctctaatattcattagaaccccattataacaatttgccgctacatggtttccaattcacagtgggttaaattgtgttacaggagacatagagtaaaaacctgatgataaacacgtagagttcacagggccggtgcaagttcttaacctcaaatattgcaaagcccttcattttgagtttaaaattttgttttatgaaatggaaagtgataatactctttgtgggcttataaattgggaagagttttggattacccctgaaacgtctctggaagcttctgttaaccctcttttttttttttttaaagtcagacaatggtatataacttttaactctcgatag

SEQ ID NO: 27 - DNA, 750 bp, synthetic construct
  source 1..750: mol_type = other DNA; note = artificial intron 14 - 750bp
gtacaaactaagcattttactcagtaactttatctgttcctagacttatagctgctaatctctaatattcattagaaccccattataacaatttgccgctacatggtttccaattcacagtgggttaaattgtgttacaggagacatagagtaaaaacctgatgataaacacgtagagttcacagggccggtgcaagttctgttcatgctctgattgggggtaaatctgcaaagcccttgtgtgttgatgattttcttaaggcctctcctgattttataactgtatagtcaaaacttttttcatttgtgtactgtggctcatttttattttgtcaggtatgtgctggtcatacttgctggatggactgtgtaaataggttttgtttatcagggtatcacacttaactgtcgtggataatcttttcctccagagagtatagatgtttaaaggaacacagctatttaatgatatggcacatcaaggtttgaacttaggtggctaataacatacctttttaaaaatgagaatgaaacatgtttataaccaaaattaacctcaaatattgcaaagcccttcattttgagtttaaaattttgttttatgaaatggaaagtgataatactctttgtgggcttataaattgggaagagttttggattacccctgaaacgtctctggaagcttctgttaaccctcttttttttttttttaaagtcagacaatggtatataacttttaactctcgatag

SEQ ID NO: 28 - DNA, 1000 bp, synthetic construct
  source 1..1000: mol_type = other DNA; note = artificial intron 14 - 1000bp
gtacaaactaagcattttactcagtaactttatctgttcctagacttatagctgctaatctctaatattcattagaaccccattataacaatttgccgctacatggtttccaattcacagtgggttaaattgtgttacaggagacatagagtaaaaacctgatgataaacacgtagagttcacagggccggtgcaagttctgttcatgctctgattgggggtaaatctgcaaagcccttgtgtgttgatgattttcttaaggcctctcctgattttataactgtatagtcaaaacttttttcatttgtgtactgtggctcatttttattttgtcaggtatgtgctggtcatacttgctggatggactgtgtaaatcaggggtctgcctcttgtggctcataaggtgattttgagtgacatggccaaagtcagccctaggcacagtcaagaagtaggcgaagtttctctttgccttcccagcttttctgtccgtgcttcttcaagagacccttccctgaagcagttgccatggtggcctcctttgccaccctctcagaaatgaccaaaatatatacattcccctctgtcctgggccaaatcttgctaaaaggagctctctttttagaggttttgtttatcagggtatcacacttaactgtcgtggataatcttttcctccagagagtatagatgtttaaaggaacacagctatttaatgatatggcacatcaaggtttgaacttaggtggctaataacatacctttttaaaaatgagaatgaaacatgtttataaccaaaattaacctcaaatattgcaaagcccttcattttgagtttaaaattttgttttatgaaatggaaagtgataatactctttgtgggcttataaattgggaagagttttggattacccctgaaacgtctctggaagcttctgttaaccctcttttttttttttttaaagtcagacaatggtatataacttttaactctcgatag

SEQ ID NO: 29 - DNA, 230 bp, synthetic construct
  source 1..230: mol_type = other DNA; note = artificial intron 16 - 230bp
gtaaaacctgtctgcctcttttcatcttaattgtttgaatatggtagtttgagatttatgagtttattttactttattaaaaacatgttcaaagattacaaatccagtttatgtgccatttctctttttaacatgtggaatttacacttcaggtttaaatttcttgtcaggccaattacagattacagtaggatatggtctgtgtatataacaactataacttgttttag

SEQ ID NO: 30 - DNA, 400 bp, synthetic construct
  source 1..400: mol_type = other DNA; note = artificial intron 16 - 400bp
gtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaattaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttag

SEQ ID NO: 31 - DNA, 750 bp, synthetic construct
  source 1..750: mol_type = other DNA; note = artificial intron 16 - 750bp
gtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaatatgcagtctgagtatttgtgaagaccttgacagccacactatgattttattatttattatttatttttatttttttgagacgggagtttcgcttttgttgcccaggctggagtgcaatggcgcaatctcggctcagagcaacctctgcctcccgggttcaagtgattctcctgccactaattttattttaactctagtgttagaggcatgtatgtcttctggtaatcagtaatagtaattcattttacctgtttgtttctcactgttttattctacttagaagaagacatgataggattgtgagtttttctctaactttggatgcagtgagatgaccagtgtgttccagttaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttag

SEQ ID NO: 32 - DNA, 1000 bp, synthetic construct
  source 1..1000: mol_type = other DNA; note = artificial intron 16 - 1000bp
gtaaaacctgtctgcctcttttcatcttaattgtttgaatatggtagtttgagatttatgagtttattttactttattaaaaacatgttcaaagattacaaatccagtttatgtgaaatattttaccttcaacattacaattgtagtcaatcttagttctttgtgacaagtatacaaataagaggatttctctagagaatatgcagtctgagtatttgtgaagaccttgacagccacactatgattttattatttattatttatttttatttttttgagacgggagtttcgcttttgttgcccaggctggagtgcaatggcgcaatctcggctcagagcaacctctgcctcccgggttcaagtgattctcctgcctcagcctcctgagtagctggaattacaggcatccactaccatgcccagctaatttttgtatttttagtagagacggggtttcaccatgttggtcaggctggtcttgaactcccaacctcaggtgagatcatacctgtgatagatataaagatgtaaattaaagtctagttagtgagttatttgagggtaaagccagaataggaaaacattttgtttctcttttcctgggcttcacaatctttggagaataactaattttattttaactctagtgttagaggcatgtatgtcttctggtaatcagtaatagtaattcattttacctgtttgtttctcactgttttattctacttagaagaagacatgataggattgtgagtttttctctaactttggatgcagtgagatgaccagtgtgttccagttaaagaagaagagtgttttaaaatcataaaccaaataaagaatcctaccttacattaatttcttgcacttcttctgttctcatctccatttctctttttaacatgtggaatttacacttcaggtttaaatttcttgtcaggccaattacagattacagtaggatatggtctgtgtatataacaactataacttgttttag

SEQ ID NO: 33 - DNA, 4441 bp, Homo sapiens
  source 1..4441: mol_type = other DNA; note = FMRP isoform 1 (full length)
ctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatgaaaaagcaaaaaa

SEQ ID NO: 34 - DNA, 4333 bp, Homo sapiens
  source 1..4333: mol_type = other DNA; note = FMRP isoform 7
acttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatg

SEQ ID NO: 35 - DNA, 4154 bp, Homo sapiens
  source 1..4154: mol_type = other DNA; note = FMRP isoform 8
ggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaa

SEQ ID NO: 36 - DNA, 4271 bp, Homo sapiens
  source 1..4271: mol_type = other DNA; note = FMRP isoform 9
acttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatgaaaaagcaaaaaa

SEQ ID NO: 37 - DNA, 4159 bp, Homo sapiens
  source 1..4159: mol_type = other DNA; note = FMRP isoform 17
ggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcact

SEQ ID NO: 38 - DNA, 3437 bp, Homo sapiens
  source 1..3437: mol_type = other DNA; note = FMRP isoform 18
gggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgca

SEQ ID NO: 39 - DNA, 4067 bp, Homo sapiens
  source 1..4067: mol_type = other DNA; note = FMRP isoform 19
ggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaa

SEQ ID NO: 40 - DNA, 877 bp, synthetic construct
  source 1..877: mol_type = other DNA; note = Beta actin promoter
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 41 - DNA, 133 bp, synthetic construct
  source 1..133: mol_type = other DNA; note = SV40 polyA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 42 - DNA, 72 bp, synthetic construct
  source 1..72: mol_type = other DNA; note = 2x HA tag (Human influenza hemagglutinin)
ggaggagggggctccggatacccctatgacgtcccggattatgcttacccatacgatgtgccggactatgct

SEQ ID NO: 43 - DNA, 735 bp, synthetic construct
  source 1..735: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatcttga

SEQ ID NO: 44 - DNA, 22 bp, synthetic construct
  source 1..22: mol_type = other DNA
agtaacgccaatagggactttc

SEQ ID NO: 45 - DNA, 22 bp, synthetic construct
  source 1..22: mol_type = other DNA
ggcgtacttggcatatgataca

SEQ ID NO: 46 - DNA, 24 bp, synthetic construct
  source 1..24: mol_type = other DNA
ttacggtaaactgcccacttggca

SEQ ID NO: 47 - DNA, 2357 bp, synthetic construct
  source 1..2357: mol_type = other DNA
gcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcc

(19) United States
(12) Patent Application Publication
(10) Pub. No.: US 2026/0297623 A1
(43) Pub. Date: Oct. 1, 2026

(54) Novel Fragile X Constructs

(71) Applicant: uniQure biopharma B.V., Amsterdam (NL)

(72) Inventors: Lodewijk Julius Anton Toonen, Amsterdam (NL); Xu Tao, Amsterdam (NL); Astrid Valles-Sanchez, Amsterdam (NL)

(73) Assignee: uniQure biopharma B.V., Amsterdam (NL)

(21) Appl. No.: 19/489,903

(22) PCT Filed: Jun. 21, 2024
(86) PCT No.: PCT/EP2024/067403

§ 371 Date: Dec. 4, 2025

(30) Foreign Application Priority Data
Jun. 23, 2023 (EP) 23181313.0

(51) Int. Cl. C12N 15/864; A61K 9/00; A61K 38/00; A61K 48/00; C07K 14/47

(52) U.S. Cl. CPC C12N 15/8645; A61K 9/0019; A61K 38/00; A61K 48/005; C07K 14/47; C12N 2750/14132; C12N 2750/14143; C12N 2750/14171; C12N 2830/34; C12N 2830/42

(57) ABSTRACT

The present invention relates to a nucleic acid construct comprising a nucleotide sequence encoding two or more Fragile X Messenger Ribonucleoprotein (FMRP) isoforms, which also finds application in the development of AAV vectors that lead to the successful (in vivo) expression of two or more FMRP isoforms and the beneficial results which are produced by the application of such therapy in terms of improved cognitive and/or behavioural symptoms of Fragile-X Syndrome.

### FIELD OF THE INVENTION

The present invention relates to the fields of medicine, molecular biology, gene therapy and insect cell culture. In particular, the invention relates to novel Fragile X constructs and methods for treating Fragile X.

### BACKGROUND OF THE INVENTION

Fragile X is a rare hereditary condition characterized by autism, anxiety, poor attention and various cognitive problems. Fragile X has an early onset and is currently diagnosed around 3 years of age, when mild motor delays are noted in affected children. During further development, various other clinical problems become apparent and children with Fragile X typically don't develop IQ scores over 70. Additionally, Fragile X is the most common inherited cause of inherited autism with boys generally more severely affected than girls, due to Fragile X being an X-linked disorder.

Fragile X is caused by the mutational expansion of a CGG trinucleotide repeat in the promoter region of the FMR1 (Fragile X Messenger Ribonucleoprotein 1) gene, located on the X-chromosome. Fragile X-associated disorders include Fragile X Syndrome (FXS), Fragile X-associated primary ovarian insufficiency (FXPOI) and Fragile X-associated tremor/ataxia syndrome (FXTAS). FXS specifically is diagnosed when the CGG trinucleotide repeat is expanded to over approximately 200 CGG repeats. This mutation leads to the promoter region being subject to DNA methylation, resulting in reduced activity of the promoter. As a result, the expression level of the FMR1 gene is strongly reduced or even completely absent in cells. FMR1 encodes the FMRP protein, which is an RNA binding protein with a broad range of cellular functions ascribed to it. Absence of the FMRP protein most overtly affects the cells in the brain, resulting in abnormal brain development and function. In healthy neuronal cells, FMRP is known to modulate the local translation of various proteins associated with synapses. As such, the absence of FMRP leads to altered synaptic plasticity, which can affect neuronal excitability, learning, memory and social behaviour.

The FMR1 gene can be expressed as many different isoforms, with over 20 different protein coding isoforms described to date (Pretto, D. I. et al 2015, J Med Genet 52, 42-52). Many of these FMR1 isoforms have been found to be expressed at various levels in brain tissue of healthy controls (Pretto, D. I. et al. 2015 supra, Fu, X. et al. 2015 Mol Med Rep 12, 1957-1962). The full length canonical FMR1 transcript (ENST00000370475.9) contains a total of 17 exons and encodes a 632 amino acid FMRP protein (Q06787-1, SEQ ID NO. 13). At the protein level there are several important domains in FMRP, such as the RNA binding domains and nuclear localization signal, that can be partially absent depending on the exact isoform. In healthy brain, the FMR1 isoforms differ mostly by alternative splicing occurring in the last 6 exons, exon 11 till 17, whereas exons 1-10 are mostly retained in the detected brain isoforms. At the protein level, part of the KH2 RNA binding domain, the nuclear export signal and RGG (arg-gly-gly box) domains are included in the region encoded by exon 11-17 region (Dockendorff, T. C. et al 2019, Mol Neurobiol 56, 711-721). For this reason, it may be postulated that the different FMRP isoforms in brain have different functions within the cell, and may be required for healthy cellular functioning since in FXS, the expression levels of FMRP, therefore the normal ratio of the different isoforms, is also disturbed. In the context of gene therapy for FXS, expressing multiple FMR1 isoforms is likely more beneficial than expressing a single FMR1 isoform in brain.

Isoform nomenclature as described in Pretto, D. I. et al. supra, is commonly used to refer to the various FMR1 isoforms. In healthy brain, the FMR1 isoform 17 (ENST00000687593.1, FMR1-223) is reported to be the highest expressed isoform, representing an approximate 40% of total FMR1 transcripts in brain. The FMR1 isoform 7 (NM_001185076, FMR-201) is the second most common expressed isoform at approximately 25% of total FMR1 transcripts. FMR1 isoforms and reported expression levels in brain are listed in Table 1 and the exon structure of these isoforms are depicted in FIG. 1.

TABLE 1
FMR1 isoforms expressed in healthy brain. Isoform nomenclature and relative
brain expression percentages as described in Pretto, D. I. et al. 2015 supra.

| | | | | Approximate |
|---|---|---|---|---|
| | | Uniprot | | brain expression |
| FMR1 isoform | Ensembl ID | protein ID | Amino acids | level |
| Isoform 1 | ENST00000370475.9 | Q06787-1 | 632 | 0.7% |
| | FMR1-205 | | | |
| Isoform 17 | ENST00000687593.1 | X5D907 or | 594 | 38.8% |
| | FMR1-223 | Q06787-2 | | |
| Isoform 7 | ENST00000218200.12 | Q06787-9 | 611 | 24.9% |
| | FMR1-201 | | | |
| Isoform 18 | ENST00000370477.5 | A8MQB8 | 582 | 10.0% |
| | FMR1-206 | | | |
| Isoform 19 | ENST00000691214.1 | A0A8I5KT56 | 569 | 9.5% |
| | FMR1-229 | | | |
| Isoform 8 | ENST00000691111.1 | A0A8I5KS01 | 599 | 5.9% |
| | FMR1-228 | | | |
| Isoform 9 | ENST00000440235.6 | Q06787-8 | 586 | 5.4% |
| | FMR1-208 | | | |

Currently, there is no treatment that effectively addresses the cognitive impairment and autistic behaviours of individuals with Fragile X syndrome. Furthermore, speculative therapies, that are limited to the expression of only one FMR1 isoform, have not provided sufficient, if any, results for the treatment of Fragile X syndrome (WO2019227219/WO2022016055). There is, therefore, a need for new treatment modalities which address the limitations of current treatments and are more closely aligned to the wild-type FMRP1 isoform expression profile. It is therefore an object of the current invention to provide a treatment for Fragile X syndrome wherein more than one isoform is provided.

### SUMMARY OF THE INVENTION

In a first aspect, there is provided a nucleic acid construct comprising a nucleotide sequence encoding two or more Fragile X Messenger Ribonucleoprotein (FMRP) isoforms, wherein the nucleic acid comprises a first artificial intron between exons 16 and 17.
In a second aspect, there is provided a recombinant adeno-associated virus (rAAV) vector comprising the nucleic construct as disclosed herein.
In a third aspect, there is provided the nucleic acid construct or the vector as disclosed herein for use as a medicament.
In a fourth aspect, there is provided the nucleic acid construct or the vector as disclosed herein for use in the treatment (or gene therapy) of a trinucleotide repeat disorder, preferably a trinucleotide CGG repeat disorder, more preferably Fragile X-associated conditions.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer or a neurologic disease, varies depending upon the manner of administration, the age, body weight, and general health of the subject. Ultimately, the attending physician or veterinarian will decide the appropriate amount and dosage regimen. Such amount is referred to as an “effective” amount, which may be determined for instance as genome copies per kilogram (GC/kg) or as GC per dose. Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatment, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” and “similarity” also means the degree of sequence relatedness between amino acid or nucleic acid sequences, as the case may be, as determined by the match between strings of such sequences. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, more preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly(A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant parvoviral proteins and/or nucleic acids, such as baculoviral vectors for expression of parvoviral proteins and/or nucleic acids in insect cells.

A “parvoviral vector” is defined as a recombinantly produced parvovirus or parvoviral particle that comprises a polynucleotide to be delivered into a host cell, either in vivo, ex vivo or in vitro. A recombinant adeno-associated virus (rAAV) vector is an example of a parvoviral vector. Herein, a parvoviral or rAAV vector refers to both the polynucleotide comprising part of the parvoviral genome (vector genome), which usually comprises at least one ITR, a promoter and a transgene or gene of interest, as well as a parvoviral or rAAV capsid in which the polynucleotide is preferably packaged. The terms “rAAV” and “AAV” are used interchangeably.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

Any reference to nucleotide or amino acid sequences accessible in public sequence databases herein refers to the version of the sequence entry as available on the filing date of this document.

### DETAILED DESCRIPTION OF THE INVENTION

The present inventors have surprisingly found that by including intronic elements that induce splicing activity in an FMR1 expression construct, multiple FMRP isoforms that are relevant to the brain can be expressed. In a first aspect, there is provided a nucleic acid construct comprising a nucleotide sequence encoding two or more, three or more, four or more, five or more, or six or more Fragile X Mental Messenger Ribonucleoprotein (FMRP) isoforms.

The term “intronic element” as used herein refers to any nucleotide sequence within a gene that is not expressed or operative in the final RNA product. An intronic element may be between non-intron sequences, exons, and may also be known as an intergenic region, referring to both the DNA sequence within a gene and the corresponding RNA sequence in RNA transcripts. At least four different introns have been identified, classified by structure, and include: spliceosomal introns; tRNA introns; self-splicing group I introns; and self-splicing group II introns. Within introns, a donor site (5′ end of the intron), a branch site (near the 3′ end of the intron) and an acceptor site (3′ end of the intron) are required for splicing. The splice donor site commonly includes an almost invariant sequence GU at the 5′ end of the intron, within a larger, less highly conserved region. The splice acceptor site at the 3′ end of the intron terminates the intron with an almost invariant AG sequence.

The term “spliceosomal introns” as used herein refers to nuclear pre-mRNA introns that are characterized by specific intron sequences located at the boundaries between introns and exons. These sequences are recognized by spliceosomal RNA molecules when splicing is initiated. Commonly, they comprise a branch point, which refers to a nucleotide sequence at the 3′ end of the intron that becomes covalently linked to the 5′ end of the intron during the splicing process, generating a branched intron. The major spliceosome splices introns containing GU at the 5′ splice site and AG at the 3′ splice site.

The term “splicing” as used herein, refers to the work of a spliceosome at a splicing site and describes the process by which introns are excised out of the primary mRNA or pre-mRNA transcripts. Following that, the exons are joined together to generate mature mRNA. Alternative splicing is commonly used in the cell to generate multiple proteins from a single gene, called isoforms. Several methods of RNA splicing occur in nature; the type of splicing depends on the structure of the spliced intron and the catalysts required for splicing to occur.

The term “alternative splicing” as used herein, refers to the process that allows a single gene to code for multiple proteins. Alternative splicing can occur in many ways, of which the most common is exon skipping. In this mode, a particular exon may be included in mRNAs under particular conditions or in particular tissues, and omitted from the mRNA in others. Splicing is regulated by trans-acting proteins (repressors and activators) and corresponding cis-acting regulatory sites (silencers and enhancers) on the pre-mRNA. However, as part of the complexity of alternative splicing, it is noted that the effects of a splicing factor are frequently position-dependent. That is, a splicing factor that serves as a splicing activator when bound to an intronic enhancer element may serve as a repressor when bound to its splicing element in the context of an exon, and vice versa. At least five modes of alternative splicing are currently recognised and include: exon skipping; mutually exclusive exons; alternative donor site; alternative acceptor site; and intron retention (as discussed by Sammeth M, et al., 2008. A general definition and nomenclature for alternative splicing events, PLOS Com. Bio. 4(8): e1000147). Alternative methods to alter splicing are also known to one skilled in the art and include the use of antisense oligonucleotides, such as Morpholinos or Peptide nucleic acids, or epigenetic modifiers.

In some embodiments, there is provided a nucleic acid construct comprising a nucleotide sequence encoding two or more, three or more, four or more, five or more, or six or more Fragile X Messenger Ribonucleoprotein (FMRP) isoforms, wherein the nucleic acid comprises an artificial intron between exons 16 and 17 and/or between exons 14 and 15. In some embodiments, there is provided a nucleic acid construct comprising a nucleotide sequence encoding two or more Fragile X Messenger Ribonucleoprotein (FMRP) isoforms, wherein the nucleic acid comprises a first artificial intron between exons 16 and 17. In some embodiments, there is provided a nucleic acid construct comprising a nucleotide sequence encoding two or more Fragile X Messenger Ribonucleoprotein (FMRP) isoforms, wherein the nucleic acid comprises a first artificial intron between exons 14 and 15. In some embodiments, there is provided a nucleic acid construct comprising a nucleotide sequence encoding two or more Fragile X Messenger Ribonucleoprotein (FMRP) isoforms, wherein the nucleic acid comprises a first artificial intron between exons 16 and 17 and a second artificial intron between exons 14 and 15.

In some embodiments, there is provided a nucleic acid construct comprising a nucleotide sequence encoding three or more Fragile X Messenger Ribonucleoprotein (FMRP) isoforms, wherein the nucleic acid comprises a first artificial intron between exons 16 and 17. In some embodiments, there is provided a nucleic acid construct comprising a nucleotide sequence encoding three or more Fragile X Messenger Ribonucleoprotein (FMRP) isoforms, wherein the nucleic acid comprises a first artificial intron between exons 14 and 15. In some embodiments, there is provided a nucleic acid construct comprising a nucleotide sequence encoding three or more Fragile X Messenger Ribonucleoprotein (FMRP) isoforms, wherein the nucleic acid comprises a first artificial intron between exons 16 and 17 and a second artificial intron between exons 14 and 15.

In some embodiments, there is provided a nucleic acid construct comprising a nucleotide sequence encoding four or more Fragile X Messenger Ribonucleoprotein (FMRP) isoforms, wherein the nucleic acid comprises a first artificial intron between exons 16 and 17. In some embodiments, there is provided a nucleic acid construct comprising a nucleotide sequence encoding four or more Fragile X Messenger Ribonucleoprotein (FMRP) isoforms, wherein the nucleic acid comprises a first artificial intron between exons 14 and 15. In some embodiments, there is provided a nucleic acid construct comprising a nucleotide sequence encoding four or more Fragile X Messenger Ribonucleoprotein (FMRP) isoforms, wherein the nucleic acid comprises a first artificial intron between exons 16 and 17 and a second artificial intron between exons 14 and 15.

In some embodiments, there is provided a nucleic acid construct comprising a nucleotide sequence encoding five or more Fragile X Messenger Ribonucleoprotein (FMRP) isoforms, wherein the nucleic acid comprises a first artificial intron between exons 16 and 17. In some embodiments, there is provided a nucleic acid construct comprising a nucleotide sequence encoding five or more Fragile X Messenger Ribonucleoprotein (FMRP) isoforms, wherein the nucleic acid comprises a first artificial intron between exons 14 and 15. In some embodiments, there is provided a nucleic acid construct comprising a nucleotide sequence encoding five or more Fragile X Messenger Ribonucleoprotein (FMRP) isoforms, wherein the nucleic acid comprises a first artificial intron between exons 16 and 17 and a second artificial intron between exons 14 and 15.

In some embodiments, there is provided a nucleic acid construct comprising a nucleotide sequence encoding six or more Fragile X Messenger Ribonucleoprotein (FMRP) isoforms, wherein the nucleic acid comprises a first artificial intron between exons 16 and 17. In some embodiments, there is provided a nucleic acid construct comprising a nucleotide sequence encoding six or more Fragile X Messenger Ribonucleoprotein (FMRP) isoforms, wherein the nucleic acid comprises a first artificial intron between exons 14 and 15. In some embodiments, there is provided a nucleic acid construct comprising a nucleotide sequence encoding six or more Fragile X Messenger Ribonucleoprotein (FMRP) isoforms, wherein the nucleic acid comprises a first artificial intron between exons 16 and 17 and a second artificial intron between exons 14 and 15.

### FMRP Isoforms

The FMR1 gene can be expressed as many different isoforms, with over 20 different protein coding isoforms described to date (Pretto, D. I. et al 2015, J Med Genet 52, 42-52). Many of these FMR1 isoforms have been found to be expressed at various levels in brain tissue of healthy controls (Pretto, D. I. et al. 2015 supra; Fu, X. et al. 2015 Mol Med Rep 12, 1957-1962). The full length canonical FMR1 transcript (ENST00000370475.9) contains a total of 17 exons and encodes a 632 amino acid FMRP protein (Q06787-1, SEQ ID NO. 13). As used herein, the nomenclature as defined by Pretto et al., supra, is commonly used in the art to identify not only the isoforms but also number the introns and exons (see Table 1). In some embodiments, the FMRP disclosed herein may be a naturally-occurring FMRP. A naturally-occurring FMRP or subunit may be from a suitable species, e.g., from a mammal such as mouse, rat, rabbit, pig, a non-human primate, or human. In some examples, the FMRP is a wild-type human protein. Naturally-occurring FMRP from various species are well known in the art and exemplary coding sequences can be retrieved from a public gene database such as GenBank (see Table 2).

TABLE 2
FMRP isoforms expressed in healthy brain.
Isoform nomenclature and relative length.

| SEQ ID | | | Length (basepairs, |
|---|---|---|---|
| NO. | Ensembl ID | Description | incl. UTR) |
| 33 | ENST00000370475.9FMR | FMRP protein isoform 1 (full | 4441 |
| | 1-205 | length) | |
| 34 | ENST00000218200.12 | FMRP protein isoform 7 | 4333 |
| | FMR1-201 | | |
| 35 | ENST00000691111.1 | FMRP protein isoform 8 | 4154 |
| | FMR1-228 | | |
| 36 | ENST00000440235.6 | FMRP protein isoform 9 | 4271 |
| | FMR1-208 | | |
| 37 | ENST00000687593.1 | FMRP protein isoform 17 | 4159 |
| | FMR1-223 | | |
| 38 | ENST00000370477.5 | FMRP protein isoform 18 | 3437 |
| | FMR1-206 | | |
| 39 | ENST00000691214.1 | FMRP protein isoform 19 | 4067 |
| | FMR1-229 | | |

The term “isoform” as used herein, refers to a protein isoform or protein variant, that originates from a single gene or gene family and is the result of genetic differences. A set of protein isoforms may be formed from alternative splicing, variable promoter usage or other post-transcriptional modifications of a single gene or gene family.

In some embodiments, there is provided the nucleic acid construct as disclosed herein, wherein alternative splicing of the first artificial intron produces two or more, three or more, four or more, five or more, or six or more mRNAs encoding the two or more Fragile X Messenger Ribonucleoprotein 1 (FMRP) isoforms. In some embodiments, there is provided the nucleic acid construct as disclosed herein, wherein alternative splicing of a second artificial intron produces two or more, three or more, four or more, five or more, or six or more mRNAs encoding the two or more, three or more, four or more, five or more, or six or more Fragile X Messenger Ribonucleoprotein (FMRP) isoforms. In some embodiments, there is provided the nucleic acid construct as disclosed herein, wherein alternative splicing of a first and a second artificial intron produces two or more, three or more, four or more, five or more, or six or more mRNAs encoding the two or more, three or more, four or more, five or more, or six or more Fragile X Messenger Ribonucleoprotein (FMRP) isoforms.

In some embodiments, there is provided the nucleic acid construct as disclosed herein, wherein alternative splicing of the first artificial intron produces six or more mRNAs encoding the two or more Fragile X Messenger Ribonucleoprotein 1 (FMRP) isoforms. In some embodiments, there is provided the nucleic acid construct as disclosed herein, wherein alternative splicing of a second artificial intron produces six or more mRNAs encoding the or six or more Fragile X Messenger Ribonucleoprotein (FMRP) isoforms. In some embodiments, there is provided the nucleic acid construct as disclosed herein, wherein alternative splicing of a first and a second artificial intron produces six or more mRNAs encoding the six or more Fragile X Messenger Ribonucleoprotein (FMRP) isoforms.

In some embodiments, there is provided the nucleic acid construct as disclosed herein, wherein alternative splicing of the first artificial intron produces five or more mRNAs encoding the six or more Fragile X Messenger Ribonucleoprotein 1 (FMRP) isoforms. In some embodiments, there is provided the nucleic acid construct as disclosed herein, wherein alternative splicing of a second artificial intron produces five or more mRNAs encoding the five or more Fragile X Messenger Ribonucleoprotein (FMRP) isoforms. In some embodiments, there is provided the nucleic acid construct as disclosed herein, wherein alternative splicing of a first and a second artificial intron produces five or more mRNAs encoding the five or more Fragile X Messenger Ribonucleoprotein (FMRP) isoforms.

In some embodiments, there is provided the nucleic acid construct as disclosed herein, wherein alternative splicing of the first artificial intron produces five or more mRNAs encoding the five or more Fragile X Messenger Ribonucleoprotein 1 (FMRP) isoforms. In some embodiments, there is provided the nucleic acid construct as disclosed herein, wherein alternative splicing of a second artificial intron produces five or more mRNAs encoding the two or more, three or more, four or more, five or more, or five or more Fragile X Messenger Ribonucleoprotein (FMRP) isoforms. In some embodiments, there is provided the nucleic acid construct as disclosed herein, wherein alternative splicing of a first and a second artificial intron produces five or more mRNAs encoding the six or more Fragile X Messenger Ribonucleoprotein (FMRP) isoforms.

In some embodiments, there is provided the nucleic acid construct as disclosed herein, wherein alternative splicing of the first artificial intron produces four or more mRNAs encoding the four or more Fragile X Messenger Ribonucleoprotein 1 (FMRP) isoforms. In some embodiments, there is provided the nucleic acid construct as disclosed herein, wherein alternative splicing of a second artificial intron produces four or more mRNAs encoding the four or more Fragile X Messenger Ribonucleoprotein (FMRP) isoforms. In some embodiments, there is provided the nucleic acid construct as disclosed herein, wherein alternative splicing of a first and a second artificial intron produces four or more mRNAs encoding the four or more Fragile X Messenger Ribonucleoprotein (FMRP) isoforms.

In some embodiments, there is provided the nucleic acid construct as disclosed herein, wherein alternative splicing of the first artificial intron produces three or more mRNAs encoding the three or more Fragile X Messenger Ribonucleoprotein 1 (FMRP) isoforms. In some embodiments, there is provided the nucleic acid construct as disclosed herein, wherein alternative splicing of a second artificial intron produces three or more mRNAs encoding the three or more Fragile X Messenger Ribonucleoprotein (FMRP) isoforms. In some embodiments, there is provided the nucleic acid construct as disclosed herein, wherein alternative splicing of a first and a second artificial intron produces three or more mRNAs encoding the three or more Fragile X Messenger Ribonucleoprotein (FMRP) isoforms.

In some embodiments, there is provided the nucleic acid construct as disclosed herein, wherein alternative splicing of the first artificial intron produces two or more mRNAs encoding the two or more Fragile X Messenger Ribonucleoprotein 1 (FMRP) isoforms. In some embodiments, there is provided the nucleic acid construct as disclosed herein, wherein alternative splicing of a second artificial intron produces two or more mRNAs encoding the two or more Fragile X Messenger Ribonucleoprotein (FMRP) isoforms. In some embodiments, there is provided the nucleic acid construct as disclosed herein, wherein alternative splicing of a first and a second artificial intron produces two or more mRNAs encoding the two or more Fragile X Messenger Ribonucleoprotein (FMRP) isoforms.

In some embodiments, the FMRP isoforms are selected from FMRP isoforms 7, 8, 9, 17, 18, and 19. In some embodiments, at least one or more, at least two or more, or at least three or more of the FMRP isoforms is selected from FMR1 isoforms 7, 8, and 9. In some embodiments, the FMRP isoforms are selected from FMRP isoforms 17, 18, and 19. In some embodiments, at least one or more, at least two or more, or at least three or more of the FMR1 isoforms is selected from FMRP isoforms 17, 18, and 19. In some embodiments, at least one or more, at least two or more, or at least three or more of the FMRP isoforms is selected from FMRP isoforms 7, 8, and 9 and at least one or more, at least two or more, or at least three or more FMRP isoforms is selected from FMRP isoforms 17, 18, and 19. In some embodiments, the FMRP isoforms comprise at least isoforms 7 and/or 17.

In some embodiments, a first FMRP isoform is selected from the group: isoform 7, isoform 8, isoform 9 and; a second FMRP isoform is selected from the group: isoform 17, isoform 18, isoform 19.

In some embodiments, at least 10%, 15%, 20%, 25%, 30%, or 40% of the total FMRP isoforms expressed is isoform 7, or a variant thereof. In some embodiments, at least 10%, 15%, 20%, 25%, 30%, 40%, 45%, 50%, 55%, 60%, 65%, or 70% of the total FMRP isoforms expressed is isoform 17, or a variant thereof. In some embodiments, at least %, 50%, 55%, 60%, 65%, 70%, 75%, 80%, 85%, 90%, 95%, or 100% of the total FMRP isoforms expressed is a combination of isoform 7 and isoform 17, or variants thereof. In some embodiments, embodiments 5%, 6%, 7%, 9%, 10%, 11%, 12%, 13%, 14%, or 15%, of the total FMRP isoforms expressed is one or more of isoforms 8, 9, 18 and 19, or variants thereof.

Identification of any specific isoform, group of isoforms or splice variants of a single gene or gene family may be achieved by using techniques known to one of skill in the art, including but not limited to PCR, qPCR, Sanger and long-read sequencing in conjunction with specific primer design, such as disclosed in (Srivastava G P., et al. Homolog-specific PCR primer design for profiling splice variants. Nucleic Acids Res. 2011 May; 39(10):e69; Xu H, et al. Detection of splice isoforms and rare intermediates using multiplexed primer extension sequencing. Nat Methods. 2019 January; 16(1):55-58).

### Artificial Intron

In some embodiments, there is provided the nucleic acid construct as disclosed herein, wherein the first and/or second artificial intron comprises a donor site; a branch site; a polypyrimidine tract; and an acceptor site. In some embodiments, the branch sequence comprises the consensus sequence, YURAC. In some embodiments, the acceptor site comprises the consensus sequence TNCAGG. In some embodiments, the branch sequence is about 20 to 25 nucleotides upstream of the acceptor site.

In some embodiments, there is provided the nucleic acid construct as disclosed herein, wherein the first and/or second artificial intron comprises GU or AU at the 5′ end. In some embodiments, there is provided the nucleic acid construct as disclosed herein, wherein the first artificial intron comprises AG or AC at the 3′ end. Identification of any one of the structural features of the first and/or artificial intron will be known by one skilled in the art, for example, by the use of sequencing analysis carried out on the intron.

In some embodiments, there is provided the nucleic acid construct as disclosed herein, wherein the first and/or second artificial intron enables the transcription of two or more, three or more, four or more, five or more, or six or more mRNAs encoding the two or more Fragile X Messenger Ribonucleoprotein 1 (FMRP) isoforms. Therefore, identification of the products of transcription may also be carried out with the use of sequencing analysis. Additionally, in addition to, or in place of, sequencing analysis, expression analysis may also be used and would be known to one of skill in the art. For example, the use of luciferase assays, GFP tagging, SAGE, microarrays and RNA sequencing may be used. Equally, mass spectrometry, ELISA and western blot may be used, in combination or in place of any one or more tools to carry out expression analysis.

The phrase “a variant thereof” as used herein with reference to nucleic acid and amino acid sequences, indicates a sequence identity of at least 70%, 75%, 80%, 85%, 90%, or 100%, such as 81%, 82%, 83%, 84%, 85%, 86%, 87%, 88%, 89%, 90%, 91, 92%, 93%, 94%, 95%, 96%, 97%, 98%, 99%, or 100% sequence identity to the referred to sequence. It would be understood by one of skill in the art that a functional equivalent is intended.

In some embodiments, there is provided the nucleic acid construct as disclosed herein, wherein the first artificial intron comprises one of SEQ ID NO. 25, 26, 27, 28, 29, 30, 31, and 32, or a variant thereof. Therefore, in some embodiments, there is provided the nucleic acid construct as disclosed herein, wherein the first artificial intron comprises a sequence of at least 70%, 75%, 80%, 85%, 90%, or 100%, such as 81%, 82%, 83%, 84%, 85%, 86%, 87%, 88%, 89%, 90%, 91, 92%, 93%, 94%, 95%, 96%, 97%, 98%, 99%, or 100% sequence identity, selected from SEQ ID NO. 25, 26, 27, 28, 29, 30, 31, and 32. In some embodiments, there is provided the nucleic acid construct as disclosed herein, wherein the first artificial intron comprises one of SEQ ID NO. 25, 26, 27, and 28, or a variant thereof. In some embodiments, there is provided the nucleic acid construct as disclosed herein, wherein the first artificial intron comprises one of SEQ ID NO. 29, 30, 31, and 32, or a variant thereof.

In some embodiments, there is provided the nucleic acid construct as disclosed herein, wherein a first artificial intron comprises one of SEQ ID NO. 25, 26, 27, 28, 29, 30, 31, and 32, or a variant thereof. In some embodiments, there is provided the nucleic acid construct as disclosed herein, wherein a first artificial intron comprises one of SEQ ID NO. 25, 26, 27, and 28, or a variant thereof, and a second artificial intron comprises one of SEQ ID NO. 29, 30, 31, and 32, or a variant thereof. In some embodiments, there is provided the nucleic acid construct as disclosed herein, wherein a first artificial intron comprises one of SEQ ID NO. 29, 30, 31, and 32, or a variant thereof, and a second artificial intron comprises one of SEQ ID NO. 25, 26, 27, and 28, or a variant thereof.

In some embodiments, the first artificial intron is introduced into a nucleotide sequence comprising one of SEQ ID NO. 1 or 2, or a variant thereof. In some embodiments, the first artificial intron is introduced into a nucleotide sequence comprising SEQ ID NO. 1, or a variant thereof. In some embodiments, the first artificial intron is introduced into a nucleotide sequence comprising SEQ ID NO. 1, or a variant thereof, wherein exon 12 is deleted. In some embodiments, the first artificial intron is introduced into a nucleotide sequence comprising SEQ ID NO. 2, or a variant thereof.

In some embodiments, the first artificial intron and second artificial intron are introduced into a nucleotide sequence comprising one of SEQ ID NO. 1 or 2, or a variant thereof. In some embodiments, the first artificial intron and second artificial intron are introduced into a nucleotide sequence comprising SEQ ID NO. 1, or a variant thereof. In some embodiments, the first artificial intron and second artificial intron are introduced into a nucleotide sequence comprising SEQ ID NO. 1, or a variant thereof, wherein exon 12 is deleted. In some embodiments, the first artificial intron and second artificial intron are introduced into a nucleotide sequence comprising SEQ ID NO. 2, or a variant thereof. Methods of insertion of the artificial intron as disclosed herein are known to one skilled in the art and include, but are not limited to, the use of restriction enzymes, zinc finger nucleases, transcription activator-like effector nucleases, CRISPR-Cas9, base editing, prime editing or any other suitable form of gene editing (Gaj T, Sirk S J, Shui S L, Liu J. Genome-Editing Technologies: Principles and Applications. Cold Spring Harb Perspect Biol. 2016 Dec. 1; 8(12):a023754).

In some embodiments, there is provided the nucleic acid construct as disclosed herein wherein the nucleotide sequence comprises one of SEQ ID NO. 5, 6, 7, and 8, or a variant thereof. In some embodiments, there is provided the nucleic acid construct as disclosed herein wherein the nucleotide sequence comprises one of SEQ ID NO. 9, 10, 11, and 12, or a variant thereof.

In some embodiments, there is provided the nucleic acid construct as described herein, wherein when the nucleotide sequence comprises a first artificial intron and second artificial intron, the nucleic acid construct comprises a nucleotide sequence comprising SEQ ID NO. 9, 10, 11, and 12, or a variant thereof. In some embodiments, the nucleotide sequences are codon optimised.

In one aspect, there is provided an artificial intron comprising a nucleotide of SEQ ID NO. 25, 26, 27, 28, 29, 30, 31, and 32, or a variant thereof. The intron as disclosed herein, induces alternative splicing when inserted 5′ of and/or 3′ of and/or between one or more exons. In some embodiments, the artificial intron is introduced into a nucleotide sequence comprising one of SEQ ID NO. 1 or 2, after exon 16 or exon 14, preferably between exons 16 and 17 or between exons 14 and 15. In some embodiments, at least two artificial introns are introduced into a nucleotide sequence comprising one of SEQ ID NO. 1 or 2, after exon 16 and exon 14, preferably between exons 16 and 17 and between exons 14 and 15. In some embodiments, the first artificial intron and/or the second artificial intron as described herein, induces alternative splicing by one or more of: partial exon skipping; complete exon skipping; mutually exclusive exons; alternative donor site; alternative acceptor site; and intron retention. In a further embodiment, the first artificial intron and/or the second artificial intron as described herein, induces alternative splicing by exon skipping.

### Expression Constructs, Host Cells and Methods for Producing an AAV Vector

The present disclosure also finds application in the development of AAV vectors that lead to the successful (in vivo) expression of two or more FMRP isoforms and the beneficial results which are produced by the application of such therapy in terms of improved cognitive and/or behavioural symptoms of FXS (in a mouse model).

In one aspect, there is provided, a recombinant adeno-associated virus (rAAV) vector comprising the nucleic construct as herein provided above.

In some embodiments, the expression construct of the FMRP isoforms is for expression in a host cell that is suitable for the production of an AAV, such as a mammalian or insect cell line as further defined below.

Thus, in some embodiments, the expression construct for the FMRP isoforms is an insect cell-compatible vector or a mammalian cell-compatible vector. A “mammalian cell-compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses. Any vector can be employed as long as it is insect cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, transduction, transfection or infection.

In some embodiments, the vector is a baculovirus, a viral vector, or a plasmid. In some embodiments, the insect cell-compatible vector is a baculovirus, i.e. the nucleic acid construct is a baculovirus-expression vector (BEV). It is well-known that baculovirus-expression vectors are particularly suitable for the transfer of nucleic acids to insect cells and methods for their use are described for example in: Summers and Smith, 1986, “A Manual of Methods for Baculovirus Vectors and Insect Culture Procedures”, Texas Agricultural Experimental Station Bull. No. 7555, College Station, Tex.; Luckow, 1991, In Prokop et al., “Cloning and Expression of Heterologous Genes in Insect Cells with Baculovirus Vectors' Recombinant DNA Technology and Applications”, 97-152; King and Possee, 1992, “The baculovirus expression system”, Chapman and Hall, United Kingdom; O'Reilly, Miller, and Luckow, 1992, “Baculovirus Expression Vectors: A Laboratory Manual”, New York; Freeman and Richardson, 1995, “Baculovirus Expression Protocols”, Methods in Molecular Biology, volume 39; U.S. Pat. No. 4,745,051; US2003148506; and WO 03/074714. Expression constructs for separate expression of the various capsid proteins in mammalian cells are e.g. disclosed in Judd et al. (Mol Ther Nucleic Acids. 2012; 1: e54). Expression constructs for separate expression of the various capsid (Cap) proteins in insect cells are disclosed in WO2022/253955. Expression constructs for separate expression of the various replicase (Rep) proteins in insect cells are disclosed in WO 2021/198508 and WO 2009/014445.

Expression constructs for expression of all three of the VP1, VP2 and VP3 capsid proteins from a single expression cassette in mammalian cells are e.g. disclosed in Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760) and Xiao et al. (1998, J. Virol. 72, 2224-2232). Expression constructs for expression of all three of the VP1, VP2 and VP3 capsid proteins from a single expression cassette in insect cells are e.g. disclosed in Urabe et al. (2002, Hum. Gene Ther. 13:1935-1943), WO2007/046703, WO2015/137802 and WO2019/016349. As will be understood, expression of all three of the VP1, VP2 and VP3 capsid protein variants as described herein, from a single coding sequence (in a single expression cassette), allows to produce AAV vectors comprising mutations/modifications as described herein in all three of its VP1, VP2 and VP3 capsid proteins.

Expression constructs for expression of all the replicase proteins from a single expression cassette in insect cells are e.g. disclosed in WO 2009/014445. As will be understood, expression of all the replicase proteins as described herein, from a single coding sequence (in a single expression cassette), allows to replicate all BEVs encoded genes and subsequently package the rAAV vector genome into the rAAV capsids such that rAAV particles containing a vector genome are produced.

In one aspect, there is provided a host cell comprising the nucleic acid construct or expression construct or vector for the expression of the FMRP isoforms as described herein. The host cell, preferably is a host cell that is suitable for the production of AAV vectors. Accordingly the host cell is a host cell that is amenable to in vitro culture, preferably at large scale. Host cell that are suitable for the production of AAV vectors are well-known in the art and will typically be a mammalian or an insect cell line. Mammalian cell lines for producing AAV vectors are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this disclosure; nor is the type of mammalian cell, i.e., fibroblast, hepatocyte, tumor cell. Mammalian cell lines for producing AAV vectors in particular include a broad range of HEK293 cell lines, of which the HEK293T cell line is preferred.

Insect cell lines for producing AAV vectors can be any cell line that is suitable for the production of heterologous proteins. Preferably the insect cell allows for replication of baculoviral vectors and can be maintained in culture, more preferably in suspended culture. In some embodiments, the insect cell allows for replication of recombinant parvoviral vectors, including rAAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and ExpresSF+® (U.S. Pat. No. 6,103,526; Protein Sciences Corp., CT, USA) (also WO 2021/198510 and WO 2022/207899).

In some embodiments, the host cell comprising the expression construct for the expression of the FMRP isoforms as described herein, further comprises nucleotide sequences for the expression of an rAAV vector. Such further nucleotide sequences typically include expression constructs for the expression of AAV Rep and/or Cap proteins in the host cell in question. Such further nucleotide sequences in addition usually include the nucleic acid construct as disclosed herein flanked by at least one AAV ITR sequence.

AAV sequences that may be used as described herein for the production of a recombinant AAV virion, i.e. an AAV vector, in insect cells can be derived from the genome of any AAV serotype. Generally, the AAV serotypes have genomic sequences of significant homology at the amino acid and the nucleic acid levels, provide an identical set of genetic functions, and produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71:6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74:8635-47). Any AAV serotype can be used as source of AAV nucleotide sequences for use in the context of the present invention. Preferably the AAV ITR sequences for use in the context of the present invention are derived from AAV1, AAV2, AAV4 and/or AAV7. Likewise, the Rep (Rep78/68 and Rep52/40) coding sequences are preferably derived from AAV1, AAV2, AAV4 and/or AAV7.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73 (2): 939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells. Modified “AAV” sequences also can be used in this context, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8, AAV9, AAV10, AAV11, AAV12 or AAV13 ITR or Rep can be used in place of wild-type AAV ITR or Rep sequences.

In one aspect, there is provided a method for producing an AAV vector virion as described herein, comprising a nucleotide sequence encoding the FMRP isoforms as described herein. The method preferably comprising the steps of: a) culturing a host cell as herein defined above under conditions such that the AAV vector is produced; and, b) optionally, one or more of recovery, purification and formulation of the AAV vector.

The AAV in the supernatant can be recovered and/or purified using suitable techniques which are known to those of skill in the art. For example, monolith columns (e.g., in ion exchange, affinity or IMAC mode), chromatography (e.g., capture chromatography, fixed method chromatography, and expanded bed chromatography), centrifugation, filtration and precipitation, can be used for purification and concentration. These methods may be used alone or in combination. In one embodiment, capture chromatography methods, including column-based or membrane-based systems, are utilized in combination with filtration and precipitation. Suitable precipitation methods, e.g., utilizing polyethylene glycol (PEG) 8000 and NH₃SO₄, can be readily selected by one of skill in the art. Thereafter, the precipitate can be treated with benzonase and purified using suitable techniques. In addition, recovery may preferably comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74:277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV9 of Clade F capsids.

In general, suitable methods for producing an AAV vector virion as described herein in mammalian or insect host cells, and means therefore (such as expression constructs for expression of AAV rep proteins), are described, for mammalian cells in: Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760), Xiao et al. (1998, J. Virol. 72, 2224-2232) and Judd et al. (Mol Ther Nucleic Acids. 2012; 1: e54), and for insect cells in: Urabe et al. (2002, Hum. Gene Ther. 13:1935-1943), WO2007/046703, WO2007/148971, WO2009/014445, WO2009/104964, WO2011/122950, WO2013/036118, WO2015/137802, WO2019/016349 and in co-pending applications EP21177449.2, PCT/EP2021/058794 and PCT/EP2021/058798, all of which are incorporated herein in their entirety.

### Expression of the Transgene

The nucleic acid construct encoding two or more FMRP isoforms as described herein, may be located such that it will be incorporated into an recombinant parvoviral (rAAV) vector formed in the insect cell. It is understood that a particularly preferred mammalian cell in which the resulting proteins are to be expressed, is a human cell.

Alternatively, or in addition as another gene product, the nucleic acid construct encoding two or more FMRP isoforms as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, supra. Furthermore, the nucleic acid construct encoding two or more FMRP isoforms as defined herein above may comprise a further nucleotide sequence encoding a polypeptide that may serve as a fail-safe mechanism that allows to cure a subject from cells transduced with the recombinant parvoviral (rAAV) vector as described herein, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31:844-849).

The nucleic acid construct encoding two or more FMRP isoforms as defined herein above for expression in a mammalian cell, further preferably comprises at least one mammalian cell-compatible expression control sequence, e.g. a promoter, that is/are operably linked to the sequence coding for the gene product of interest. Many such promoters are known in the art (see Sambrook and Russel, 2001, supra). Constitutive promoters that are broadly expressed in many cell-types, such as the CMV, SV40, JeT, CAG and PGK promoters, may be used. However, more preferred will be promoters that are inducible, tissue-specific, cell-type-specific, or cell cycle-specific. For example, for liver-specific expression (as disclosed in PCT/EP2019/081743) a promoter may be selected from an a1-anti-trypsin promoter, a thyroid hormone-binding globulin promoter, an albumin promoter, LPS (thyroxine-binding globin) promoter, HCR-ApoCII hybrid promoter, HCR-hAAT hybrid promoter and an apolipoprotein E promoter, LP1, HLP, minimal TTR promoter, FVIII promoter, hyperon enhancer, ealb-hAAT. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185:2101-10). Even more preferred, for neuron-specific expression a promoter may be selected from a neuron-specific enolase (NSE) promoter, platelet-derived growth factor (PDGF) promoter, platelet-derived growth factor B-chain (PDGF-(3) promoter, synapsin or synapsin-1 (Syn or Syn-1) promoter, methyl-CpG binding protein 2 (MeCP2) promoter, Ca^{+/}calmodulin-dependent protein kinase II (CaMKII) promoter, metabotropic glutamate receptor 2 (mGluR2) promoter, neurofilament light (NFL) or heavy (NFH) promoter, β-globin minigene nβ2 promoter, preproenkephalin (PPE) promoter, enkephalin (Enk) promoter and excitatory amino acid transporter 2 (EAAT2) promoter. For astrocyte-specific expression a promoter may be selected from glial fibrillary acidic protein (GFAP) and EAAT2 promoters. For oligodendrocyte-specific expression the myelin basic protein (MBP) promoter a promoter may be selected. A particularly preferred promoter for expression of the nucleic acid construct encoding two or more FMRP isoforms in the peripheral and/or central nervous system is the CBh promoter (Gray et al., 2011, Hum. Gene Ther. 22:1143-1153). An endogenous promoter may also be selected, for instance, the FMR1 promoter may be selected to provide increased cellular specificity of FMRP expression (Jiang et al., 2022, Mol Ther Methods Clin Dev. 27:246-258).

Various modifications of the nucleotide sequences as defined above, including e.g. the wild-type parvoviral sequences, for proper expression in insect cells is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions, e.g. codon-optimization, are known to the skilled artisan which could increase yield of the encoded proteins. These modifications are within the scope of the present disclosure.

In some embodiments, the nucleic acid construct encoding two or more FMRP isoforms is operably linked to a promoter, optionally operably linked to a poly-A signal. In some embodiments, the nucleic acid construct encoding two or more FMRP isoforms is operably linked to a promoter and Poly-A signal. In some embodiments, the nucleotide sequence encoding two or more FMRP isoforms is operably linked to a promoter, optionally operably linked to a poly-A signal and flanked by at least one AAV Inverted Terminal Repeat (ITR). In some embodiments, the nucleotide sequence encoding two or more FMRP isoforms is operably linked to a promoter, a poly-A signal and flanked by at least one AAV Inverted Terminal Repeat (ITR).

### Compositions

In one aspect, there is provided a composition comprising an AAV vector virion as described herein, i.e. an AAV vector virion comprising at least one AAV capsid protein variant as described herein.

In some embodiments, there is provided a composition comprising an AAV vector virion as described herein and a suitable excipient, such as a buffer, stabilizer, antioxidant etc. In one particular embodiment these compositions are used to transduce cells in vitro or ex vivo, in which case the excipients will need to be compatible with cell culture.

In some embodiments, the compositions are used for treatment of (human) subjects. For that purpose there is provided a pharmaceutical composition comprising an AAV vector virion as described herein and at least one pharmaceutically acceptable carrier. In the case of AAV gene delivery vehicles a pharmaceutical composition typically comprise physiological buffers, such as e.g. PBS, comprising further stabilizing agents such as e.g. sucrose. Such compositions are compatible with and suitable and intended for use in subsequent intravenous, intrathecal, intraparenchymal, intravitreal, subretinal administration or for use in organ-targeted vascular delivery such as intraportal or intracoronary delivery or isolated limb perfusion.

### Uses

In one aspect, there is provided the use of the nucleic acid construct or vector as described herein, or an AAV virion or composition comprising the AAV vector virion.

In some embodiments, there is provided the nucleic acid construct or vector as described herein, or an AAV virion or composition comprising the AAV vector virion for use as a medicament. In one embodiment, there is the nucleic acid construct or vector as described herein, or an AAV virion or composition comprising the AAV vector virion, is for use (as a medicament) in the treatment (or gene therapy) of a trinucleotide repeat disorder, preferably a trinucleotide CGG repeat disorder, more preferably a Fragile X-associated conditions.

In one aspect, there is provided the nucleic acid construct or vector as described herein, or an AAV virion or composition comprising the AAV vector virion, for use (as a medicament) in gene therapy. In one embodiment, there is provided the nucleic acid construct or vector as described herein, or an AAV virion or composition comprising the AAV vector virion, for administration to a subject in need of gene therapy.

In some embodiments, the gene therapy is for the treatment of a disease defined herein above (e.g. a trinucleotide repeat disorder, preferably a trinucleotide CGG repeat disorder, more preferably Fragile X-associated conditions, most preferably FXS). In some embodiments, the gene therapy is for the treatment of a CGG trinucleotide repeat wherein there are more than 200 CGG repeats. In some embodiments, the gene therapy is for the treatment of Fragile X Syndrome.

The present invention has been described above with reference to a number of exemplary embodiments as shown in the drawings. Modifications and alternative implementations of some parts or elements are possible, and are included in the scope of protection as defined in the appended claims.

### DESCRIPTION OF THE FIGURES

FIG. 1. Most common FMR1 gene isoforms in human brain. Exon structure of the FMR1 gene isoforms that are the most abundant in cells of the brain using Isoform 1 as reference sequence (full length FMR1 ENST00000370475.9). Relative percentages of each isoform are listed in Table 1. The listed brain isoforms all lack exon 12. Differences between the brain specific FMR1 gene isoforms are caused by various combinations of alternative splicing events in exon 15 and exon 17.

FIG. 2. Overview of structural FRM1 sequence layout summarizing the expression constructs described herein. FMR1 exons 1-13 are not depicted, but the parent isoform used here is isoform 7 ENST00000218200.12, thus lacking exon 12 compared to full length FMR1. A) Expression construct structure with intronic sequence element between exon 16 and exon 17 (i.e. intron 16), theoretically capable of forming FMR1 isoform 7 and 17. B) Expression construct structure with intronic elements introduced between exon 14 and exon 15 (intron 14) and between exon 16 and exon 17 (intron 16). The potential FMRP isoforms that can be expressed based on the combination of possible splice events are depicted, with their relative expected percentage abundance in brain tissue.

FIG. 3. PCR confirmation of FMR1 exon 17 splicing from transfected cells. HEK293 cells were transfected with the indicated plasmids, RNA was isolated and RT-PCR performed using primers from exon 14 to the 3′ HA tag of the FMR1 constructs. Size separation on agarose gel showed product sizes of isoform 7 (SEQ ID NO. 2) and isoform 17 (SEQ ID NO. 4). For the FMR1 intron 16 splicing construct (SEQ ID NO. 5) both isoforms were detected, confirming correct splicing and expression.

FIG. 4. Westernblot analysis of FMRP protein expressed from exon 17 splicing constructs. HEK293 cells were transfected with the indicated plasmids, protein was isolated and westernblot analysis performed. FMRP protein expressed from the plasmids was confirmed through probing with HA tag antibody (top panel). Total FMRP, including endogenously expressed variants, were additionally confirmed by anti-FMRP probing (middle panel). SEQ ID NO. 2 and 4 conditions were included as protein size references for FMRP isoform 7 and 17 respectively, but the size difference between these proteins is not discernible through westernblot analysis. Equal input was confirmed through probing of beta actin (bottom panel).

FIG. 5. PCR confirmation of FMR1 exon 15 and 17 splicing from transfected cells. HEK293 cells were transfected with the indicated plasmids, RNA was isolated and RT-PCR performed using primers from exon 14 to the 3′ HA tag of the FMR1 constructs. For size reference, cells transfected with plasmid SEQ ID NO. 2, 3 and 4, corresponding to FMR1 isoforms 7, 9 and 17 respectively, as well as the combination of all 3. Multiple isoforms for FMR1, likely corresponding to isoforms 7, 9 and 17, are expressed from constructs SEQ ID NO. 9, 10, 11 and 12. This confirms alternative splicing and expression of multiple isoforms from these plasmids.

FIG. 6. Westernblot analysis of FMRP protein expressed from exon 15 and 17 splicing constructs. HEK293 cells were transfected with the indicated plasmids, protein was isolated and westernblot analysis performed. FMRP protein expressed from the plasmids was confirmed through probing with HA tag antibody (top panel). Total FMRP, including endogenously expressed variants, were additionally confirmed by anti-FMRP probing (middle panel). SEQ ID NO.s 2, 3 and 4 conditions were included as protein size references for FMRP isoform 7, 9 and 17 respectively. The arrows on the right hand side of the panels indicate the height of FMRP isoform 7 or 17 (top arrow) and isoform 8, 9, 18 or 19 (bottom arrow). Equal input was confirmed through probing of beta actin (bottom panel).

FIG. 7. qPCR assays confirm multiple isoform expression from FMR1 gene expression vectors. A) Taqman probe based assays with different specificities were implemented to quantify level of differential splicing of exon 17. Assay locations are depicted below exon structure of the 2 classes of FMR1 isoforms. Approximate amplicon and probe location (black boxes) are depicted. B) qPCR analysis of ID1. C) qPCR analysis of ID2. D) qPCT analysis of ID3.

FIG. 8. Long read RNA sequencing based assessment of vector expressed FMR1 gene isoform ratios. Oxford nanopore based long read sequencing was performed on RNA from HEK293T cells transfected with the indicated constructs. Reads were filtered on presence of the HA tag sequence, so only the vector derived FMR1 transcripts are detected. Reads were subsequently filtered to a minimum length of 1000 base pairs to ensure coverage of the relevant exon junctions of the FMR1 transcript. These reads were then mapped to the 6 FMR1 gene isoforms listed to assess splicing and transcript ratios from the different vectors. The most predominant isoforms detected are isoform 7 and 17, in line with the PCR analysis. Results are depicted as percentage of FMR1 gene isoform per vector.

FIG. 9. qPCR confirmation of vector uptake and FMR1 expression in rostral cortex of AAV injected mice.

AAVs carrying SEQ ID NO. 6, 9 and 43 were tested by injection in wildtype mouse brain. A) The level of vector uptake in rostral cortex was quantified through qPCR analysis. Successful uptake was confirmed for all three AAVs used here. AAVs were not titer matched prior to injection, and the difference in genome copy number observed in cortex of the mice corresponded to the dose of the injected AAV stock concentration. B) The expression of total FMR1 transcript was quantified through qPCR. FMR1 upregulation is reported as fold change over reference SEQ ID NO. 43, corresponding to GFP expression control group, thus representing wildtype murine FMR1 level. Between 5 and 50 fold upregulation of FMR1 was observed in cortex, and the fold upregulation corresponded well to the amount of vector DNA present in tissue, as depicted in C.

FIG. 10. PCR confirmation of FMR1 exon 15 and 17 splicing in wildtype brain. RT-PCR flanking the expected FMR1 splice sites was performed using primers from exon 14 to the 3′ HA tag of the FMR1 exogenous transcript. Multiple FMR1 isoforms are detected in the treated mice, with animals treated with SEQ ID NO. 6 showing the expected two isoforms. Animals treated with SEQ ID NO. 9 showed the presence of three distinct isoforms, thus corresponding to the transfection experiment performed in HEK cells. FMR1 isoform 7 and 17 plasmid was used for size reference in the PCR.

FIG. 11. Western blot confirmation of FMRP protein expressed in mouse brain. In vivo FMRP protein expression in nine mice treated with the indicated expression constructs was confirmed through western blot. Probing with HA-tag antibody, specific to the exogenous FMRP protein, confirmed full length FMRP protein expression in mice treated with SEQ ID NO. 6 and SEQ ID NO. 9 (top panel). Similar to in vitro testing in HEK cells, the different FMRP isoforms resulting from the alternative splicing could be confirmed in animals treated with SEQ ID NO. 9. For animals treated with SEQ ID NO. 6, a single FMRP band could be discerned again similar to results obtained in HEK cells. Brain tissue from an FMR1 knockout animal treated with SEQ ID NO. 4 is depicted in the first lane as reference and to confirm assay specificity. Probing for the GFP protein, expressed from the SEQ ID NO. 43 construct, confirmed expression of the GFP protein in the corresponding treatment group.

### EXAMPLES

### Introduction

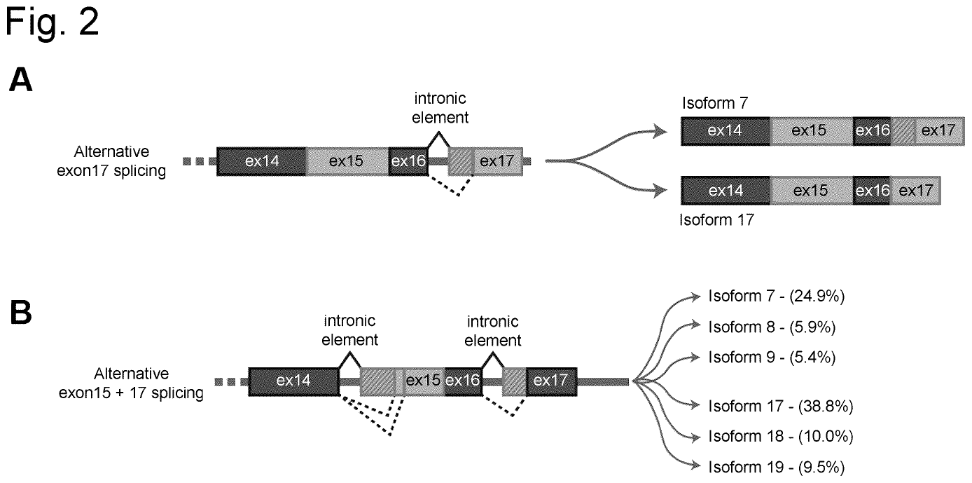

The FMR1 coding regions described herein include intronic elements capable of inducing splicing activity in order to express multiple FMR1 protein isoforms that are relevant to the brain. Intronic elements were included between exon 14 and 15 as well as between exon 16 and 17 of the FMR1 gene following numbering and nomenclature of Ensembl ID ENST00000370475.9 FMR1 exon 15 contains 2 known alternative splice acceptor signals and exon 17 contains 1 alternative splice acceptor signal (FIGS. 2A and B). As such, exon 15 can occur in at least 3 different lengths, whereas exon 17 can occur in at least 2 different lengths. Variation in exon 15 and exon 17 lengths, i.e. alternative splicing, is responsible for the majority of FMR1 isoforms described in brain. Indeed, alternative splicing of exon 15 and exon 17 in FMR1 can differentiate between the 6 isoforms that represent approximately 95% of the total FMR1 expression level in healthy brain. By inclusion of intronic elements, these FMR1 isoforms can be expressed from a single expression construct. Alternative splicing in exon 17 can differentiate between FMR1 isoform 7 and 17, which together are expected to represent an approximate 64% of total FMR1 transcript level in brain Pretto, D. I. et al 2015, J Med Genet 52, 42-52. An additional 31.5% of FMR1 isoforms in brain is differentiated by alternative splicing events in exon 15. For this reason, two main construct classes are described here, one class containing an intronic sequence element introduced between exon 16 and 17 (FIG. 2A) and a class with an additional intronic sequence introduced between exon 14 and 15 (FIG. 2B).

### Methods

### Generation of FMR1 Gene Expression Constructs

FMR1 gene expression constructs containing SEQ ID NO.s 1-8 were generated by standard gene synthesis of FMR1 cDNA sequences at Azenta/Genewiz (Leipzig, Germany). Synthesized DNA sequences were subcloned by Genewiz using NheI and EcoRV restriction sites to a PuC based custom vector backbone (SEQ ID NO 47) already containing a promoter (SEQ ID NO 40) and SV40 polyA sequence (SEQ ID 41). The full subcloned PuC construct thus contained the promoter, FMR1 coding region exon 1-17 (excluding exon 12), and polyA signal. The vector backbone further contained origin of replication and Ampicillin selection marker to facilitate amplification of the vector in E. coli. In a second round of gene synthesis, SEQ ID NO.s 9-12 containing various intron 14 length variants were synthesized and subcloned to parent vector isoform 7 (SEQ ID NO. 2) using internal restriction enzymes PsyI and BamH1. In this manner, FMR1 isoform 7 expression constructs containing various length variations of intron 14 and intron 16 were generated for testing in vitro. All sequences were verified by restriction digest and Sanger sequencing by Azenta.

### Plasmid Transfections and FMR1 Expression

FMR1 expression plasmids containing SEQ ID NO.s 1-12 were transfected in HEK293T cells using lipofection following standard procedures. RNA and protein was isolated after two days to assess FMR1 expression levels and identification of different FMRP isoforms arising from the plasmid constructs.
PCR and qPCR Analysis
Isolated RNA was subjected to reverse transcription and obtained cDNA subjected to PCR and qPCR analysis. A reverse primer binding in the DNA sequence encoding the HA tag was implemented to specifically amplify exogenous FMR1 sequences. Forward primers in either exon 14 or the 3′ HAtag sequence (SEQ ID NO.s 20 and 21) were used in the PCR reaction in order to amplify the FMR1 region where splicing events were expected to occur. PCR products were then separated on agarose gel to confirm size, and isolated for Sanger sequencing confirmation. For qPCR analysis, premade Taqman assay Hs00924547_m1 and Hs00924539_m1 (ThermoFisher Scientific, Darmstadt, Germany) and custom Taqman assay (forward exon 16, reverse HA-tag, probe exon 17 second splice acceptor) were implemented. Hs00924539_m1 is suitable to assess all FMR1 isoforms described here, due to amplification region of exon 2 and 3. Assay Hs00924539_m1 was found to be insensitive for Isoform 17, likely due to probe binding in the 5′ region of exon 17, and thus serves as a control to detect FMR1 isoforms containing full length exon 17 (i.e. Isoform 1, 7, 8 and 9). The custom Taqman qPCR was instead specific for isoforms showing splicing of exon 17 (i.e. isoform 17, 18 and 19). The combination of these various primer sets allows for relative quantification of the two different classes of FMR1 isoforms, with or without alternative exon 17 splicing.
For analysis of vector DNA levels in mouse brain tissue, DNA was isolated and qPCR primers (SEQ ID NO.s 44 and 45) and corresponding probe (SEQ ID NO. 46) against the promoter region that was common to the three AAVs being tested was used for qPCR. Absolute quantification of vector DNA copies in brain tissue was performed by comparison to a plasmid based standard line, and copy numbers are reported as viral genome copies per total μg of DNA. The total level of FMR1 mRNA in mouse brain tissue was quantified using premade Taqman Hs00924539_m1, which also detects the murine FMR1 transcript in wildtype mice. Premade Taqman reference gene assays Mm99999915_g1 (GAPDH), Mm00446968_m1 (HPRT) and Mm00446953_m1 (GUSB) were used for normalization of expression level.

### Western Blotting Analysis

Isolated protein fractions of the plasmid transfected HEK293T cells were separated on SDS-PAGE gel and transferred to nitrocellulose membrane. FMRP protein was detected using rabbit anti-FMRP antibody ab17722 (Abcam, Cambridge, UK) or mouse anti HA-tag antibody 6E2 (Biolegend, San Diego, CA, USA). Followed by 800CW or 680CW antibodies (Licor, Lincoln, NE, USA) and detected on an Odyssey CLx imager. Band intensities, corresponding to FMRP expression levels, were quantified using Image Studio Lite software (Licor). Quantification of FMPR levels in mouse brain tissue were performed using the same methods, but rabbit anti GAPDH antibody PA1-987 (ThermoFisher) was used for input control and mouse anti GFP antibody GF28R (ThermoFisher) was used to confirm expression of the GFP protein in the control group.

### In Vivo AAV Testing in Mice

The expression constructs for SEQ ID NO. 6, 9 and 43 were subcloned to baculo virus generation constructs containing ITR (inverted terminal repeats) and used to infect sf9 insect cells for AAV generation. Obtained AAVs were purified and titers determined following standard procedures. Wildtype mouse pups were then injected intracerebroventricularly on the day of birth or at one day old to improve AAV distribution throughout the brain. A total of 4 μl of AAV with a stock concentration between 2.0×10¹³ and 5.0×10¹³ was injected bilaterally, for a total dose of between 8.0×10¹⁰ and 2.0×10¹¹ genome copies of AAV per animal. The animals were sacrificed 6 weeks later and brain tissue was isolated and used for subsequent analysis of FMR1 mRNA and protein expression as described.

### Long Read RNA Sequencing

Isolated RNA from the transfected HEK293T cells was used for Oxford Nanopore based long read sequencing. RNA samples were DNAase treated first to remove vector DNA as much as possible. RNA was shipped to Baseclear (Leiden, the Netherlands) for library preparation and sequencing. Direct cDNA Sequencing Kit (SQK-DCS109), followed by Native Barcoding Kit 96 V14 (SQK-NBD114.96). Sequencing was performed on a promethion 2 Solo machine (Oxford Nanopore technologies, Oxford UK). Between approximately 5 and 6.5 million reads were obtained per sample, which were then filtered for presence of the 72 nucleotide HA-tag sequence (SEQ ID NO. 42), which is specific to the exogenous, vector expressed FMR1 transcripts. Reads were further filtered by minimum size selection of 1000 base pairs. Filtered reads were first aligned to the full length FMR1 transcript (SEQ ID NO. 1) in order to detect all possible variants. The filtered reads also mapped against the specific FMR1 isoforms (SEQ ID NOs. 34-39) in CLC Genomics version 22.0.2Obtained number of read counts per FMR1 isoform transcript were then reported.

### Example 1

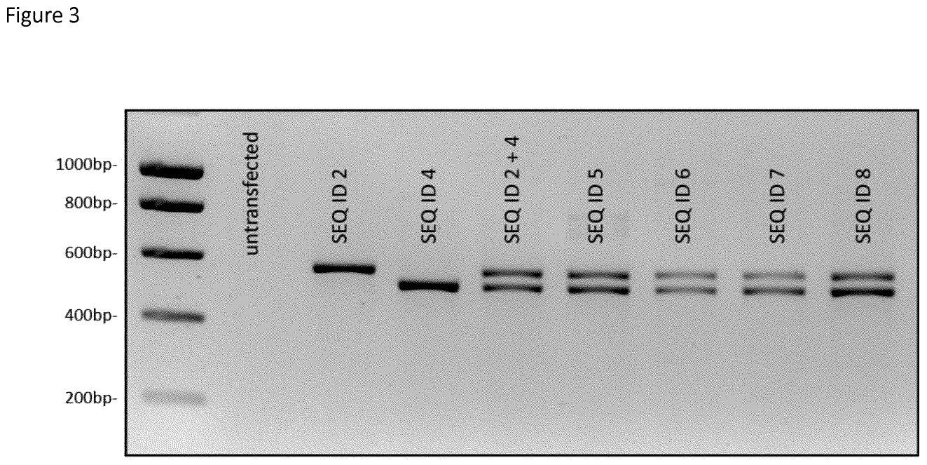

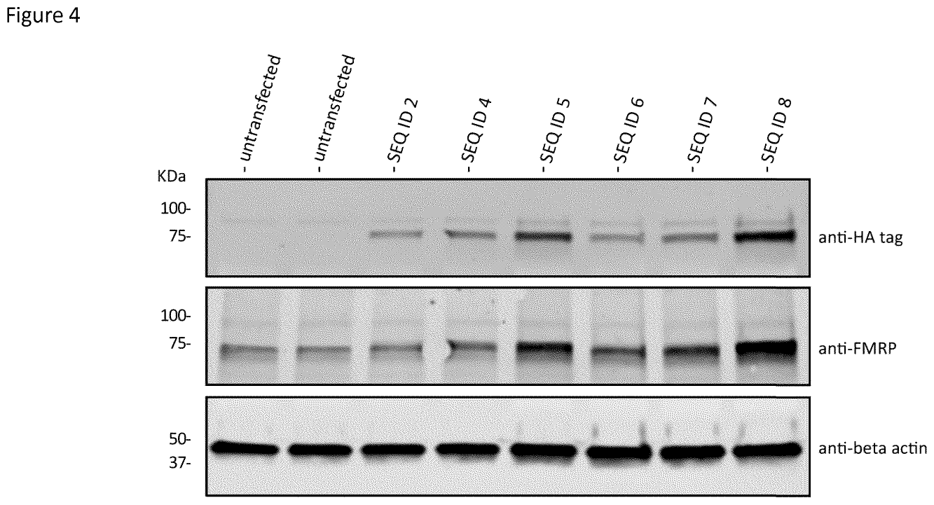

FMR1 Splicing Confirmation Following Transfection with Plasmid SEQ ID NO.s 5-8 (Intron 16 Variants)
Plasmids containing FMR1 expression sequences as indicated were transfected in HEK293T cells using lipofection. RT-PCR using primers (SEQ ID NO.s 20 and 21) were used to specifically amplify the exogenous FMR1 expressed cDNA from exon 14 until the 3′ HA-tag. The PCR product was separated on agarose gel to detect different sizes of FMR1 transcripts corresponding to the expected isoform sizes (FIG. 3). The height of the observed bands of the FMR1 construct SEQ ID NO. 5, SEQ ID NO. 6, SEQ ID NO. 7 and SEQ ID NO. 8 treated cells is in correspondence with those observed for cells treated with SEQ ID NO. 2 and 4, thus corresponding to FMR1 isoform 7 and 17 respectively. The expected PCR product sizes for FMR1 isoform 7 (SEQ ID NO. 2) is 508 basepairs, and 457 basepairs for FMR1 isoform 17 (SED ID 4). The PCR products were isolated from gel and subjected to Sanger sequencing to conclusively confirm presence of FMR1 isoform 7 and 17. This experiment thus confirms that the various intron 16 lengths used here successfully led to expression of FMR1 isoform 7 and 17 RNA from the vector SEQ ID NO. 5, SEQ ID NO. 6, SEQ ID NO. 7 and SEQ ID NO. 8.
Protein was also isolated from the HEK cells transfected with SEQ ID NO. 2, SEQ ID NO. 4, SEQ ID NO. 5, SEQ ID NO. 6, SEQ ID NO. 7 and SEQ ID NO. 8 to detect FMRP protein expression from the plasmids. As all expression constructs contain a C-terminal HA Tag (Human influenza-hemagglutinin) (SEQ ID NO. 41), an antibody specific for this tag was implemented as well to detect the FMRP protein originating from the expression vector. Successful FMRP protein expression was confirmed for all the tested conditions of plasmid transfected HEK cells (FIG. 4). FMRP protein expression was observed with both HA-tag and anti-FMRP antibodies, hence confirming the bands corresponded to exogenously expressed FMRP protein. The two FMRP proteins corresponding to isoform 7 (SEQ ID NO. 14) and isoform 17 (SEQ ID NO. 15) consist of 612 and 595 amino acids respectively, excluding the HA tag. It is not expected that this approximate 3% size difference is discernible by westernblotting, and indeed cells transfected with plasmid SEQ ID NO. 2 and SEQ ID NO. 4, corresponding to FMRP isoforms 7 and 17, yielded proteins of identical size on westernblot analysis. Similarly, one protein band was observed for SEQ ID NO. 5-8 transfected cells (FIG. 4).

### Example 2

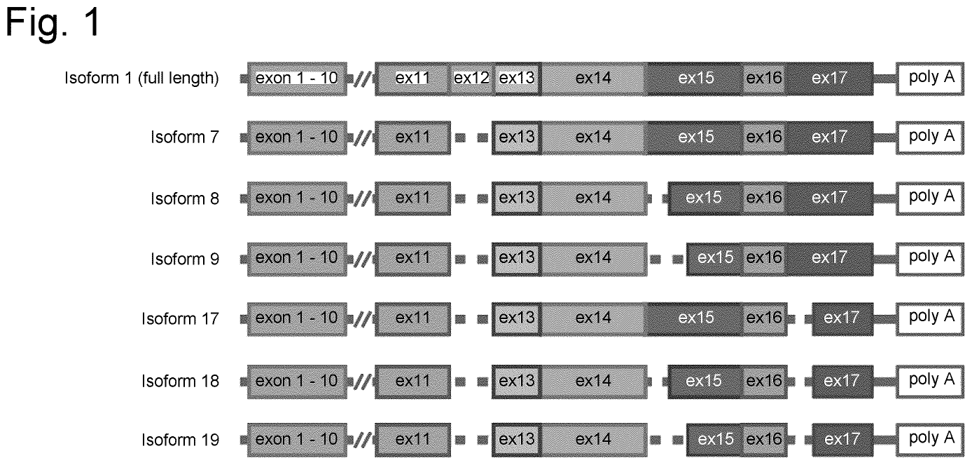

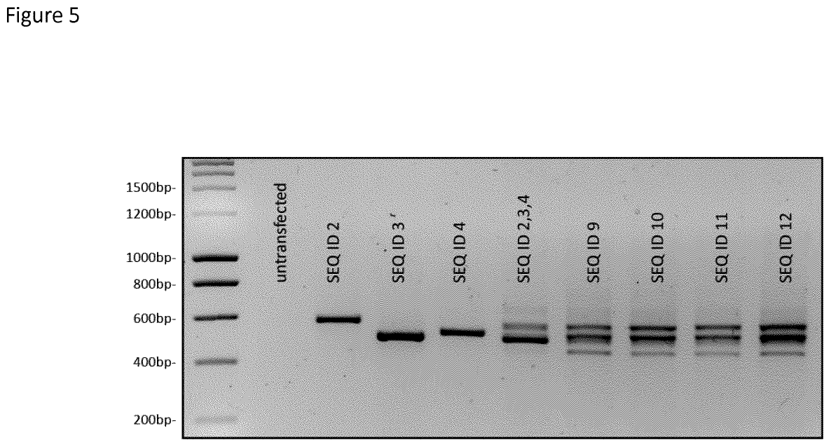

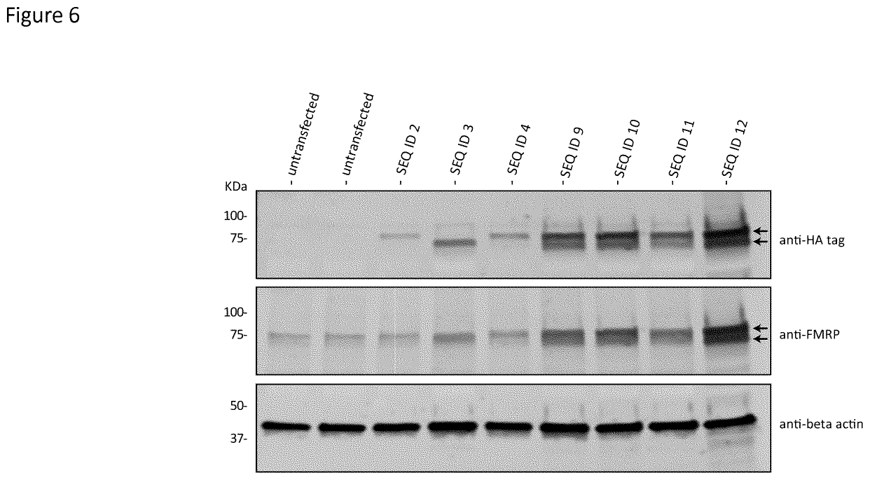

FMR1 Splicing Confirmation Following Transfection with SEQ ID NOs. 9-12 (Intron 14 and Intron 16 Variants)
Similar to the process described in example 1, plasmids corresponding to SEQ ID NO. 9-12 were transfected in HEK cells and RT-PCR analysis for exogenous FMR1 RNA performed to confirm transcript expression and confirm presence of expected splicing of the FMR1 construct. For plasmids SEQ ID NO. 9-12, intron 14 and 16 variants are included. Intron length 16 was 400 bp, equivalent to SEQ ID NO. 6, while intron lengths of intron 14 varied from 230 bp to 1000 bp. Depending on the combination of splicing events, these expression constructs have the potential to generate splicing variants FMR1 isoforms 7, 8, 9, 17, 18 and 19 (FIG. 1), due to alternative splicing of exons 15 and 17. Together these isoforms are expected to represent over ~95% of brain described FRM1 isoforms Pretto, D. I. et al 2015, J Med Genet 52, 42-52RT-PCR analysis revealed that at least 3 distinct FMR1 isoforms were expressed at RNA level (FIG. 5).
Protein was isolated from the HEK cells transfected with plasmids SEQ ID NO.s 2, 3, 4, 9, 10, 11 and 12 containing a promoter (SEQ ID NO. 40), an HA tag sequence (SEQ ID NO. 42) and a polyA signal (SEQ ID NO. 41) in order to confirm protein expression and detect potential FMRP protein isoforms. Probing for the HA-tag confirmed exogenous FMRP protein expression in the transfected cells. In this case, two separate proteins were identified based on different size for cells transfected with plasmids SEQ ID NO. 9, 10, 11 and 12 (FIG. 6, top panel). This confirms that the alternatively spliced RNA expressed from these constructs is translated into at least two FMRP protein isoforms. Probing with anti-FMRP antibody (FIG. 6, middle panel) detected the same two bands, confirming the results obtained with the HA tag antibody. Quantification of the two bands, as indicated with the arrows, showed that the top band was between 1.6 and 2.1 fold higher expressed than the lower band. The top band corresponds to FMRP isoform 7 or 17, whilst the lower band can correspond to FMRP isoform 8, 9, 18 or 19, which are not discernible based on protein size through this westernblot analysis.

### Example 3

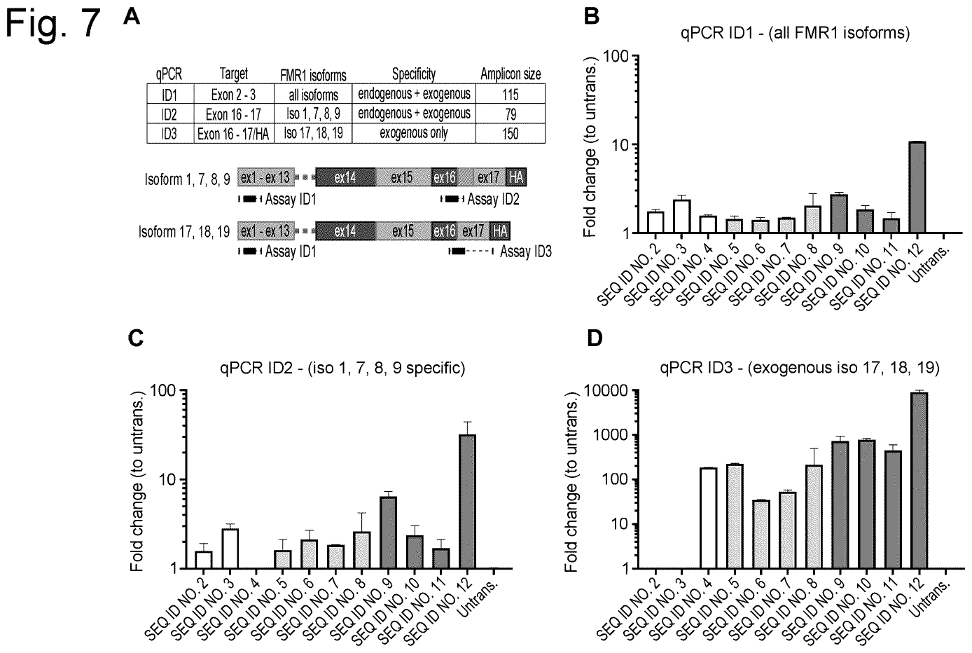

qPCR Detection of FMR1 Isoform Levels
Due to differential splicing of the FMR1 constructs, it is expected that the various FMR1 isoforms are expressed at different levels from the plasmid construct. qPCR assays were selected and designed in order to detect the various main classes of brain expressed FMR1 isoforms. These two main classes of isoforms differ in alternative splicing of exon 17 (FIGS. 1 and 2). Hence, qPCR assays were designed targeting sequencing specific to the 5′ region of exon 17 that is susceptible to alternative splicing. Three qPCR assays were used; one specific to all FMR1 isoforms, one specific for Isoform 1, 7, 8, 9 and others containing full length exon 17, and a third assay specific to the exon junction that occurs after exon 17 alternative splicing (FIG. 7A).
RT-qPCR was performed on RNA obtained from transfected HEK cells as in example 1 and 2. qPCR assays as depicted in FIG. 7A were used to determine an approximate fold expression level of the different classes of FMR1 isoforms with or without alternative exon 17 splicing. For all constructs described here, an upregulation of total FMR1 expression was observed (FIG. 7B). All FMR1 construct variants containing either 1 or 2 intronic elements (SEQ ID NO. 5-12), upregulation of FMR1 variants with and without alternative exon 17 splicing was detected (FIGS. 7C and D). This confirms all constructs tested here can generate at least two FMR1 isoforms from a single construct. The ratios between total FMR1 and the two different classes of FMR1 isoforms detected with these qPCR assays showed only subtle differences

### Example 4: FMR1 Isoform Detection Based on Long Read Sequencing

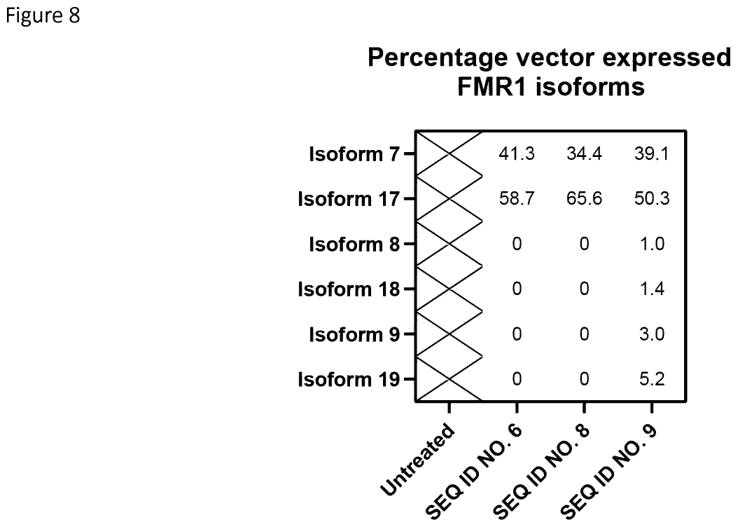

In the examples above, the FMR1 isoforms expressed following transfection of the constructs was assessed using PCR amplification and Sanger sequencing of products. To expand the analysis and obtain a more comprehensive and unbiased detection of isoforms expressed from the constructs, RNA from transfected HEK cells was used for Oxford Nanopore based long read sequencing. This technology is capable of obtaining sequencing reads sufficiently long to cover all known mRNA variants of FMR1, and allows for more objective quantification of each specific isoform. Total RNA samples from HEK293T cells transfected with: SEQ ID NO. 6, 8 and 9 were used for the long read sequencing. Obtained reads were filtered for presence of the 72 nt HA tag (SEQ ID NO. 41), which is specific to the exogenous plasmid expressed FMR1 mRNA molecules. These reads were than mapped against a list of known FMR1 isoforms (isoforms 7, 8, 9, 17, 18, and 19) in order to determine presence and ratios between these isoforms.
The percentage of different FMR1 isoforms matched with the results as assessed by PCR. The majority of reads mapped to isoforms 7 and 17 (FIG. 8). For the variant with one intron (SEQ ID 6) approximately 41% of FMR1 transcripts correspond to isoform 7 and approximately 59% correspond to isoform 17. The longer intron 16 variant (SEQ ID 8) appears to shift splicing more towards isoform 17.

### Example 5: In Vivo Proof of Mechanism of AAV9-Splicing Constructs

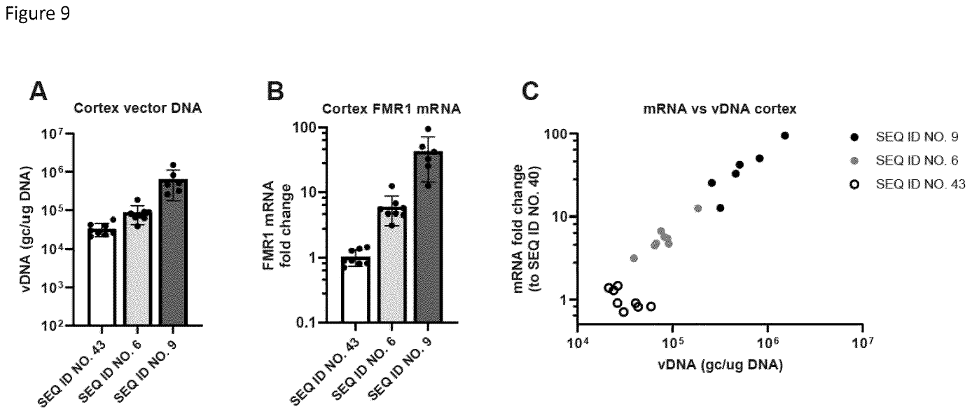

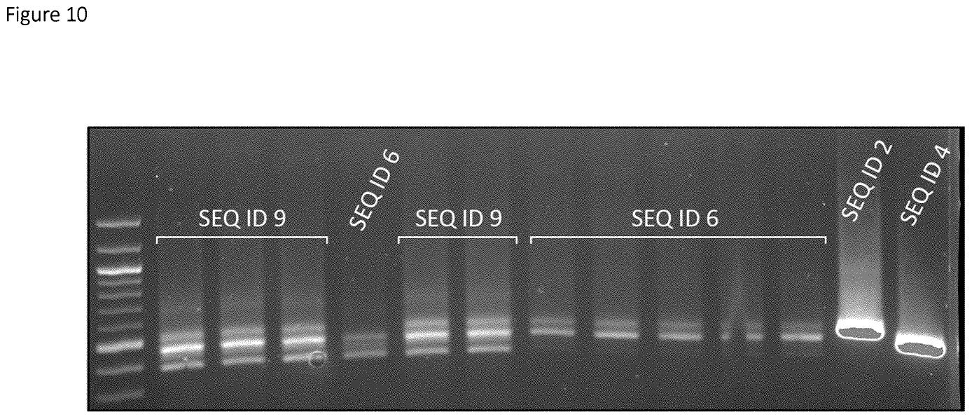

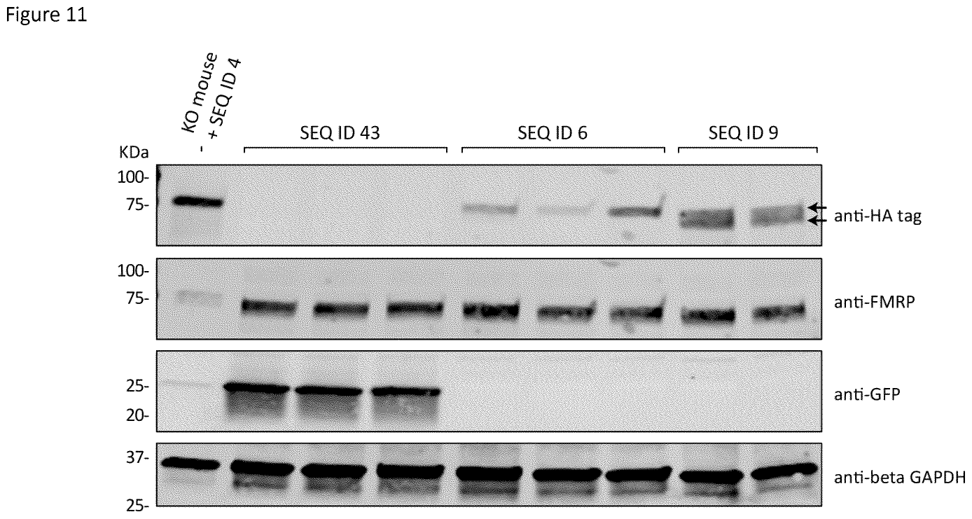

To assess the in vivo viability of the constructs, AAVs were generated for two FMR1- and one GFP expression control construct corresponding to SEQ ID NOs 6, 9 and 40 respectively. Purified AAVs were injected intracerebroventricularly in newborn wildtype mouse pups and cortex tissue of the brain was examined after a 6 weeks in life period. Successful distribution and uptake of the AAVs was confirmed through qPCR analysis of vector genome copies, with approximate levels between 1.0×10⁴ and 1×10⁶ of vector genomes per μg of DNA detected (FIG. 9A). The corresponding expression of FMR1 mRNA was subsequently confirmed with qPCR for total FMR1, and up to 50-fold upregulation over the murine FMR1 transcript was observed (FIG. 9B). This confirmed successful FMR1 expression from the constructs in mouse brain, and a good correlation between the level of vector DNA and corresponding FMR1 upregulation was observed (FIG. 9C).
PCR covering the expected splicing events that could occur in the FMR1 expression variants was then performed similar to shown in the HEK cells. The expected presence of two isoforms, corresponding in size to FMR1 isoforms 7 and 17 for SEQ ID NO. 6 treated mice was indeed observed. Similarly, presence of at least three distinct FMR1 isoforms for animals treated with SEQ ID NO. 9 (FIG. 10).
The presence of the FMRP protein isoforms was then examined through westernblot on cortex tissue of the mice. Full length FMRP protein was indeed detected through probing for the C-terminal HA-tag (FIG. 11). Two distinct FMRP protein isoforms could be detected in SEQ ID NO. 9 treated animals, and one FMRP protein band was observed in SEQ ID NO. 6 treated animals, similar to results obtained in HEK cells (FIG. 4). This confirms that the splicing pattern on FMR1 mRNA originating from the FMR1 expression constructs functions in mice similar to in vitro experiments in HEK cells.

1. A nucleic acid construct comprising a nucleotide sequence encoding two or more Fragile X Messenger Ribonucleoprotein (FMRP) isoforms, wherein the nucleic acid comprises a first artificial intron between exons 16 and 17.

2. A nucleic acid construct according to claim 1, wherein alternative splicing of the first artificial intron produces two or more mRNAs encoding the two or more FMRP isoforms.

3. The nucleic acid construct according to claim 1, wherein the nucleotide sequence comprises a second artificial intron between exons 14 and 15.

4. A nucleic acid construct according to claim 3, wherein alternative splicing of the second artificial intron produces two or more mRNAs encoding the two or more FMRP isoforms.

5. The nucleic acid construct according to claim 1, wherein the first artificial intron comprises SEQ ID NO. 29, 30, 31, 32, or a variant thereof.

6. The nucleic acid construct according to claim 1, wherein the second artificial intron comprises SEQ ID NO. 25, 26, 27, 28, or a variant thereof.

7. The nucleic acid construct according to claim 1, wherein the nucleotide sequence does not comprise exon 12.

8. The nucleic acid construct according to claim 1, wherein the nucleotide sequence comprises SEQ ID NO. 5, 6, 7, 8, or a variant thereof.

9. The nucleic acid construct according to claim 1, wherein the nucleotide sequence comprises SEQ ID NO. 9, 10, 11, 12, or a variant thereof.

10. The nucleic acid construct according to claim 6, wherein the two or more FMR1 isoforms comprise isoform 7 and/or isoform 17.

11. The nucleic acid construct according to claim 7, wherein the two or more FMR1 isoforms comprise isoform 7 and/or isoform 17 and/or;

12. The nucleic acid construct according to claim 7, wherein the two or more FMR isoforms comprise isoform 7 and/or isoform 8 and/or isoform 9 and/or isoform 17 and/or isoform 18 and/or isoform 19.

13. The nucleic acid construct according to claim 12, wherein a first FMR isoform is selected from the group:
isoform 7, isoform 8, isoform 9;
and a second FMR isoform is selected from the group:
isoform 17, isoform 18, isoform 19,
preferably isoforms 7 and 17.

14. The nucleic construct according to claim 1, wherein the nucleotide sequence encoding two or more FMRP isoforms is operably linked to a promoter, optionally operably linked to a poly-A signal, and flanked by at least one AAV Inverted Terminal Repeat (ITR)

15. An recombinant adeno-associated virus (rAAV) vector comprising the nucleic construct according to claim 12.

16.-17. (canceled)

18. A method for treating of a trinucleotide repeat disorder in a subject in need thereof, the method comprising administering the nucleic acid construct according to the subject.

19. The method according to claim 18, wherein the trinucleotide repeat disorder is a trinucleotide CGG repeat disorder.

20. The method according to claim 19, wherein the disorder is a Fragile X-associated condition.

21. The nucleic acid construct according claim 7, wherein preferably the sequence comprises SEQ ID NO. 1, 2, or a variant thereof.
